(19) United States
(12) Patent Application Publication
(10) Pub. No.: US 2026/0086092 A1
(43) Pub. Date: Mar. 26, 2026

(54) Sexually Transmitted Infection Testing from Human Dried Blood Spots Collected Remotely

(71) Applicant: Blackfly Investments, LLC, dba Molecular Testing Labs, Vancouver, WA (US)

(72) Inventors: Huilan Yao, Vancouver, WA (US); Marissa Shumaker, Vancouver, WA (US); Thea Jacobson Rosewood, Vancouver, WA (US); Charles Sailey, Vancouver, WA (US); Adam Blackwell, Vancouver, WA (US)

(21) Appl. No.: 18/897,667

(22) Filed: Sep. 26, 2024

(51) Int. Cl. G01N 33/569; G01N 33/543

(52) U.S. Cl. CPC G01N 33/56988; G01N 33/54366; G01N 33/56927; G01N 33/56994; G01N 2333/035; G01N 2333/16; G01N 2333/186; G01N 2333/20; G01N 2333/22; G01N 2333/295

(57) ABSTRACT

System and method for remote self-collection of a blood sample for assessment at a lab to detect presence of specific sexually transmitted infections (STIs). An at-home collection kit may include an ability to detect chlamydia (CT), gonorrhea (GC), bacterial vaginosis (BV), candida vaginitis (CV) and Trichomonas vaginalis (TV), HSV-2, syphilis, HIV, HBsAg, an HCV. The screening of samples for the presence of antigens and/or antibodies to infectious agents is a primary laboratory tool for the diagnosis of infections of the above viruses and bacterium. Distributing a specimen collection kit containing dried blood spot (DBS) cards can eliminate the hurdles of visiting clinics or hospitals and reduce stigma. DBS samples collected on filter paper, such as GE Whatman™ 903 protein saver cards, offer several advantages over on-site phlebotomist collection. The DBS cards and collection devices are especially useful for individuals who live in remote areas where healthcare resources may be limited.

### BACKGROUND

As techniques and methodologies for medical testing and diagnostics advance, more consumers are turning to “at-home” collection kits that may involve collecting a blood/tissue/urine sample using a collection device without the assistance of a physician/medical staff. In a typical blood collection procedure, a patient will induce blood flow into a dedicated collection medium. Blood collection devices may be used to capture a sample and then the sample is packaged for mailing or transit to a remote testing facility. After testing the “at home” collected sample, results of testing or diagnostics may then be communicated to the patient using standard confidentiality protocols. An area where “at-home” collection kits may be useful is in the area of sexually transmitted infections.

Sexually transmitted infections (STIs), also referred to as sexually transmitted diseases (STDs), are caused by passing various pathogenic microorganisms from one person to another through sexual contact. Some examples include chlamydia, gonorrhea, genital herpes, human papillomavirus, syphilis, viral hepatitis, and human immunodeficiency virus (HIV). Some of these STIs may not result in severe symptoms, such as an infection of herpes simplex virus-2 (HSV-2), however, some can lead to morbidity, such as acquired immune deficiency syndrome (AIDS) caused by HIV infection. Even without signs or symptoms, STIs can be spread to sexual partners.

The CDC estimates that there are about 20 million new infections in the United States each year, costing the American healthcare system nearly $16 billion in direct medical costs alone. Among those newly infected, young people within the age range of 15-24, are particularly affected and account for 50% of all new STI. Among the various ways of preventing STIs, early screening and treatment are critical to protect an individual's health and prevent transmission to others.

To make the STI screening test easier and more accessible to the general population, as discussed herein, a self-collection kit for detecting STIs is needed. Studies have shown that when compared to physician collection, people are more willing to provide samples if they can be self-collected and administrated in a familiar environment. In addition, at-home collection will save individuals time from making appointments and/or visiting clinics Further, research indicates that individuals are more inclined to provide samples when they can self-collect them in a familiar environment, as opposed to physician collection. Moreover, at-home collection not only saves time by eliminating the need for appointments and clinic visits but also enhances the privacy of personal health information.

### BRIEF DESCRIPTION OF THE DRAWINGS

Embodiments of the subject matter disclosed herein in accordance with the present disclosure will be described with reference to the drawings, in which:

FIG. 1 is a diagram view of an objective dried blood spot (DBS) collection device being used in a process for collecting capillary blood from a human according to an embodiment of the subject matter disclosed herein.

FIG. 2 is a diagram of a system that facilitates the detection of sexually transmitted infections from human dried blood spots collected remotely according to an embodiment of the subject matter disclosed herein.

FIG. 3 is a method diagram of preparing an instrument for an assay for detection of sexually transmitted infections from human dried blood spots collected remotely according to an embodiment of the subject matter disclosed herein.

FIG. 4 is a method diagram of a DBX protocol for preparing an elution for an instrument prepared for an assay to detect of sexually transmitted infections from human dried blood spots collected remotely according to an embodiment of the subject matter disclosed herein.

FIGS. 5-8 are method diagrams of specific DBX protocols for preparing an elution for an instrument prepared for an assay to detect the presence of sexually transmitted infections from human dried blood spots collected remotely according to embodiments of the subject matter disclosed herein.

Note that the same numbers are used throughout the disclosure and figures to reference like components and features.

### DETAILED DESCRIPTION

The subject matter of embodiments disclosed herein is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

Embodiments will be described more fully hereinafter with reference to the accompanying drawings, which form a part hereof, and which show, by way of illustration, exemplary embodiments by which the devices described herein may be practiced. These devices may, however, be embodied in many different forms and should not be construed as limited to the embodiments set forth herein; rather, these embodiments are provided so that this disclosure will satisfy the statutory requirements and convey the scope of the subject matter to those skilled in the art.

By way of an overview, the systems and methods discussed herein are directed to the self-collection of a blood sample remotely for assessment at a lab after transporting to detect presence of specific sexually transmitted infections (STIs). An at-home collection kit may include a device for utilizing molecular diagnostic techniques to analyze a female sample for detecting chlamydia (CT), gonorrhea (GC), bacterial vaginosis (BV), candida vaginitis (CV) and Trichomonas vaginalis (TV) or a male sample for detecting CT, GC, and TV. Testing may also be utilized to detect the HSV-2, syphilis, HIV, HBsAg, and HCV. The screening of samples for the presence of antigens and/or antibodies to infectious agents is a primary laboratory tool for the diagnosis of infections of the above viruses and bacterium.

Serological test involves the use of plasma or serum collected venously, requiring a visit to a hospital or an on-site testing facility with phlebotomists. However, distributing a specimen collection kit containing dried blood spot (DBS) cards can eliminate the hurdles of visiting clinics or hospitals and reduce stigma. DBS samples collected on filter paper, such as GE Whatman™ 903 protein saver cards, offer several advantages over on-site phlebotomist collection, including convenience, reduced healthcare resource utilization, and increased accessibility. In addition, there are a simple means of sample collection for the general public and are also easy to transport and store. Finally, the DBS cards and collection devices are especially useful for individuals who live in remote areas where healthcare resources may be limited.

The Clinical Laboratory Improvement Amendments (CLIA), passed in 1988, establishes quality standards for all laboratory testing to ensure the accuracy and reliability of patient test results regardless of where the test was performed. The CLIA regulations include a requirement for validating the performance of laboratory-developed tests. The purpose is to summarize the performance of these enzyme immunoassays for several sexually transmitted infections (STIs) using self-collected samples on automated liquid handler ELISA system, including Hamilton ELISA system and the Dynex DSX™ Automated ELISA System and to show that they are now validated for diagnostic usage. The combination of the “at-home” DBS self-collection and the remote preparation (e.g., laboratory preparation remote form the collection location) and analysis meets with these established standards.

The system and methods described herein are proven to test blood for the presence of STIs including Human Immunodeficiency Virus (HIV), Herpes Simplex Virus (HSV-2), syphilis, and Hepatitis C Virus (HCV) through the specimen type of dried blood via a DBS card. The workflow includes sample collected by patient at home via DBS card and finger prick kit, sent through mail to a laboratory for processing. The method continues by depositing punched chips from the received DBS card into a unique elution to be prepared into a format that can be run using a DSX system. Once an instrument is prepared, an assay is performed to determine the qualitative outcomes (negative/non-reactive or positive/reactive) of the aforementioned STIs.

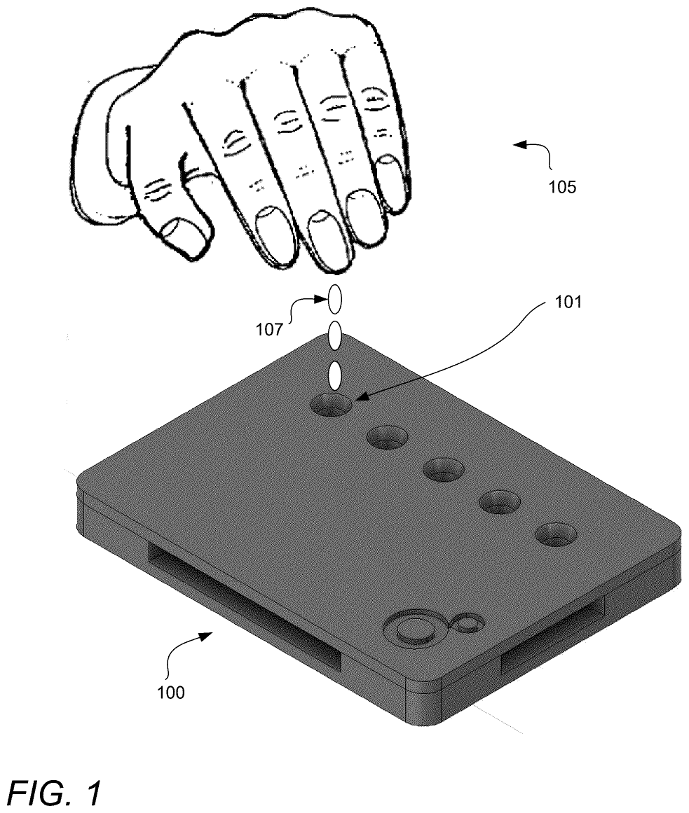

Such a collection procedure and analysis at laboratory has several advantages. A first advantage is that this procedure allows “at home” and self-collection insomuch as the patient avoids barriers to care like location, stigma, and/or cost. Another advantage is that a finger stick collection (by the patient) is extremely less invasive and can be done without a physician or nurse. Finally, the process is repeatable and can be carried out with a high degree of certainty across several different infections. These and other advantages will become readily apparent when considered in conjunction with FIGS. 1-4 and the detailed description that follows below.

FIG. 1 is a diagram view of an objective dried blood spot (DBS) collection device 100 being used in a process for collecting capillary blood from a human according to an embodiment of the subject matter disclosed herein. In this view, one can see a human hand 105 of a human patient dripping blood 107 (e.g., after being lanced) such that capillary blood 107 may drip down into, in this view, a first collection aperture 101 of the device 100. Inside the DBS collection device is DBS collection card 215 (unseen in this view) that may become saturated with the dripping blood at the specific collection points. The patient may move the lanced finger on the patient's hand 105 to the next collection aperture for saturated the next target spot on the DBS collection card 215, and so on. The objective DBS collection device 100 and related DBS card 215 are the respective subjects of U.S. patent application Ser. Nos. 18/380,030 and 18/603,536 which are each incorporated herein by reference for all purposes.

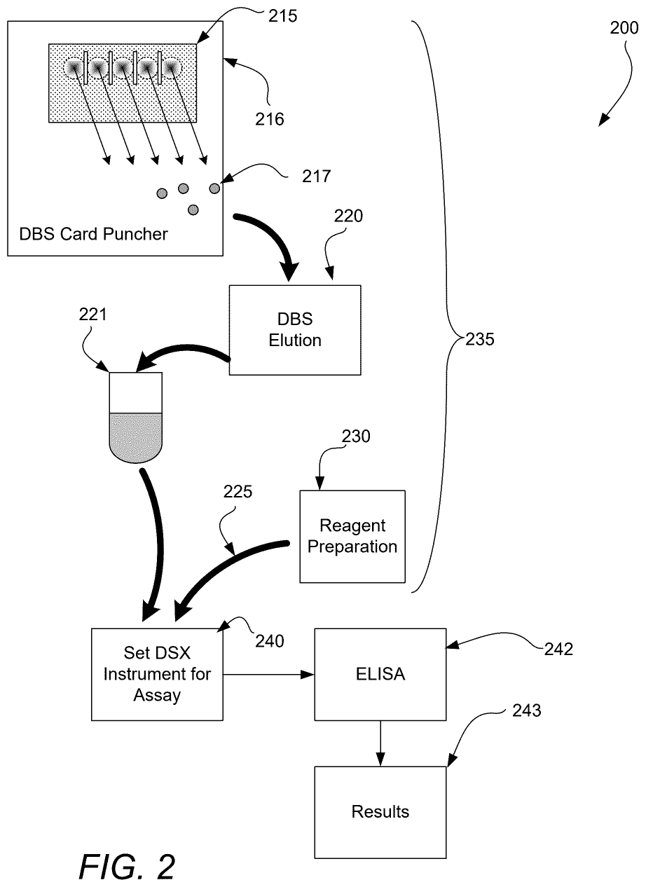

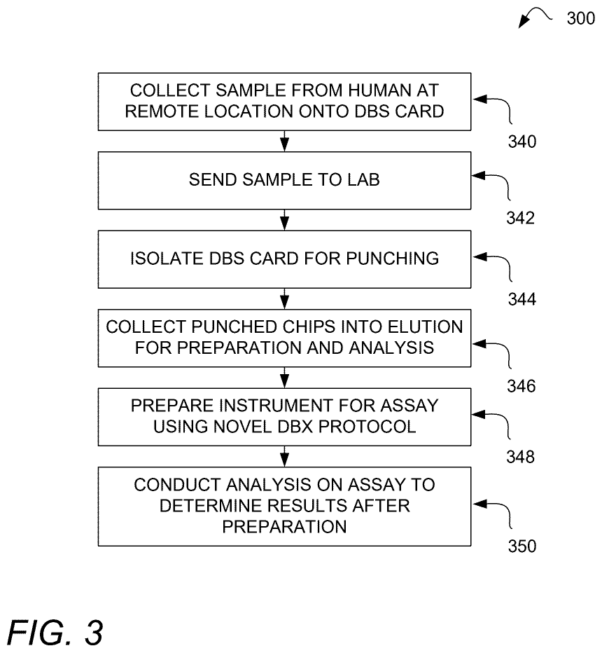

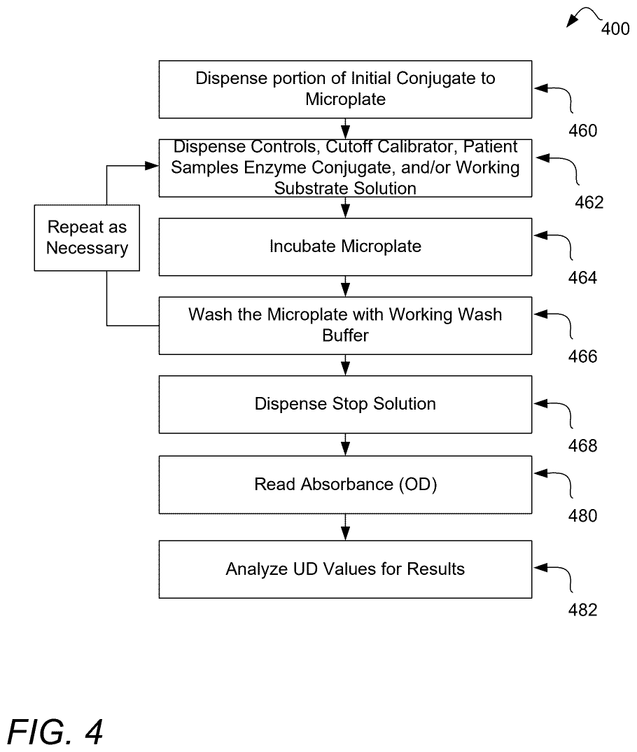

In other embodiments, the collection device 100 may be a different collection device such as a GE Whatman™ 903 collection card. As is discussed further below, the collection card 215 may be packaged in a flat package that may be easily mailed via standard mailing services to a lab for further processing and analysis. Such processing is discussed further below with respect to FIGS. 2-4 and it viable for “at-home” collection of human sample material because of this ease of shipping without the bulk of a vial holding any such fluid sample. That is, privacy and convenience are achieved with the procedures discussed below specifically because “at-home” collection is possible. Once sample are discreetly received the system of FIG. 2 and the methods of FIGS. 3-4 may be used to determine specific diagnostic results as desired or required. In these embodiments, the specifical goal of these procedures is to detect STIs.

FIG. 2 is a system diagram for a system 200 to facilitate detection of sexually transmitted infections from human dried blood spots collected remotely according to an embodiment of the subject matter disclosed herein. The blood-saturated area of DBS specimen 215 is cut into circular chips 217 (or discs) by manual or automated puncher 216. These chips 217 are placed into an elution tube or 96-well plate 220, and an elution buffer is added into the tubes/plate to elute the analytes. An eluent is well-suited for follow-on procedures involving a protocol for assay instrument preparation. One such preparation protocol is a DSX™ protocol. DSX™ is an automated open system capable of performing up to four assays per microplate simultaneously, providing optimized efficiency and speed. The DSX™ protocol uses a synchronized system to eliminate microplate drift and realizes consistency across different microplate incubators. In other embodiments, the DSX™ protocol is not used.

When the chips 217 are in the DBS elution, each sampled DBS chip 217 is completely submerged into the elution buffer, and the sample may be incubated for a period of time up to 4 hours at a temperature range of between 15° C. and 37° C. In one embodiment, the incubation is two hours at ambient temperature. Then, after incubation, the elution buffer containing the antigen/antibodies (from the sample DBS chips 217) may be transferred into a sample tube 221 for analysis without the underlying chips 217. Once the incubated elution (now without any chips) is in a suitable sample tube 221, the protocol may continue to prepare the elution for use with an instrument in a DSX™ protocol.

In this protocol, specific procedures are followed to create and elution suitable for assay analysis on a prepared instrument. These procedures include adding reagents 225 such as PBS (Phosphate Buffered Saline) containing mild detergent, such as 0.05% Tween-20, BIO-RAD GS HIV Combo Ag/Ab EIA (BIO-RAD #26217), Trinity Biotech Trep-Sure™EIA (Trinity #TS-96), ORTHOR HCV EIA 3.0 Reagent Kit (BIO-RAD #930740), HerpeSelect® 2 ELISA IgG (FOCUS Diagnostics #EL0920G). Reagents may be prepared at a reagent preparation station 230 by a lab technician following procedures for one or more specific DSX™ protocols. Further, one or more specific DSX™ protocols may include establishing positive controls, negative controls, and cutoff calibrators that may be part of a commercial ELISA assay kit. These positive controls, negative controls, and cutoff calibrators are discussed further below with respect to sections on examples.

As such, these specific procedures can prepare the elution for a DSX instrument 231 to be used in a standard operating procedure (SOP) for an assay to detect presence of a specific analytes—such as an STI as discussed herein.

An assay is an investigative (analytic) procedure in laboratory medicine for qualitatively assessing the presence of a target entity. The detected entity is often called the analyte, the measurand, or the target of the assay. The analyte can be proteins (antigens and/or antibodies). An assay usually aims to qualitatively measure an analyte's intensive property and express it in the relevant measurement unit (e.g., optical density).

Assays are becoming more prevalent in modern medical, environmental, pharmaceutical, and forensic technology. Other businesses may also employ them at the industrial, curbside, or field levels. Assays in high commercial demand have been well investigated in the research and development sectors of professional industries. They have also undergone generations of development and sophistication. In some cases, they are protected by intellectual property regulations such as patents granted for inventions. Such industrial-scale assays are often performed in well-equipped laboratories and with automated organization of the procedure, from ordering an assay to pre-analytic sample processing (sample collection, necessary manipulations e.g., spinning for separation, aliquoting if necessary, storage, retrieval, pipetting, aspiration, and the like). Analytes are generally tested in high-throughput auto-analyzers, and the results are verified and automatically returned to ordering service providers and end-users.

As an instrument (e.g., a microplate) is prepared, additional steps for affecting the elution may be carried out to ensure best practices for realizing proper results. Generally speaking, these preparation procedures are collectively referred to in FIG. 2 as preparation 235. Such preparation steps may include incubating the elution for specified time over specified temperature. Additionally, the preparation may include rocking and/or shaking the elution over time and specific speeds and/or velocities. Once all preparation steps are concluded a prepared instrument 240 is generated.

Once an instrument 240 is prepared via the procedures described above, the instrument 240 is now ready for ELISA assay. ELISA is “Enzyme Linked Immuno-Sorbent Assay” and is a standard procedure for analysis based upon how an instrument is prepared. The ELISA system 242 produces usable analysis and results 243. Specific aspects of the protocol and analysis are discussed below on a per-STI basis.

FIG. 3 is a method diagram of preparing an instrument for an assay for detection of STI analytes from human DBS cards collected remotely according to an embodiment of the subject matter disclosed herein. In this method, collected samples from a human using a DBS card are collected remotely at step 340. The collected sample may be blood, urine, or the like. After collection, the DBS card is received at a laboratory after remote collected at step 342. The received sample may include a sample-laden DBS card for punching that tis ready to be punched at step 344. That is, a DBS card punching device may create sample chips form the saturated areas of the received DBS card. These sample chips are punched at step 346 and placed into the elution for further processing.

Once the elution receives punched chips, a protocol to prepare an instrument with the elution for unique assay is undertaken at step 348. Specific steps related to this preparation are discussed further below with respect to FIG. 4. Once an instrument is prepared, the assay analysis procedure may be undertaken to produce results at step 350.

FIG. 4 is a generic method diagram of a protocol for preparing an elution for an instrument prepared for an assay to detect of sexually transmitted infections from human dried blood spots collected remotely according to an embodiment of the subject matter disclosed herein. In an embodiment, the elution may be initially set using sample chips from a received and already-sampled DBS card that has been punched. The elution, previously prepared using sample chips, may undergo a loading of reagents as discussed previously. The prepared conjugate may be sampled and loaded to a microplate at step 460. The microplate may then be loaded with controls, cutoff calibrators, and additional patent samples as required by a specific assay at step 462. Once initially loaded, the microplate may undergo an incubation at step 464 before undergoing a working wash buffer at step 466. Loading 462, incubating 464 and washing 466 may be repeated as necessary per specific protocol. Once finally prepared, the microplate may be used with an ELISA system to read Absorbance values (OD) at step 480 so as to inform results at step 482.

As discussed herein, the procedures and preparations may be used for several different applications on a per-test basis for various STI detection procedures. Examples of these specific test procedures are discussed next.

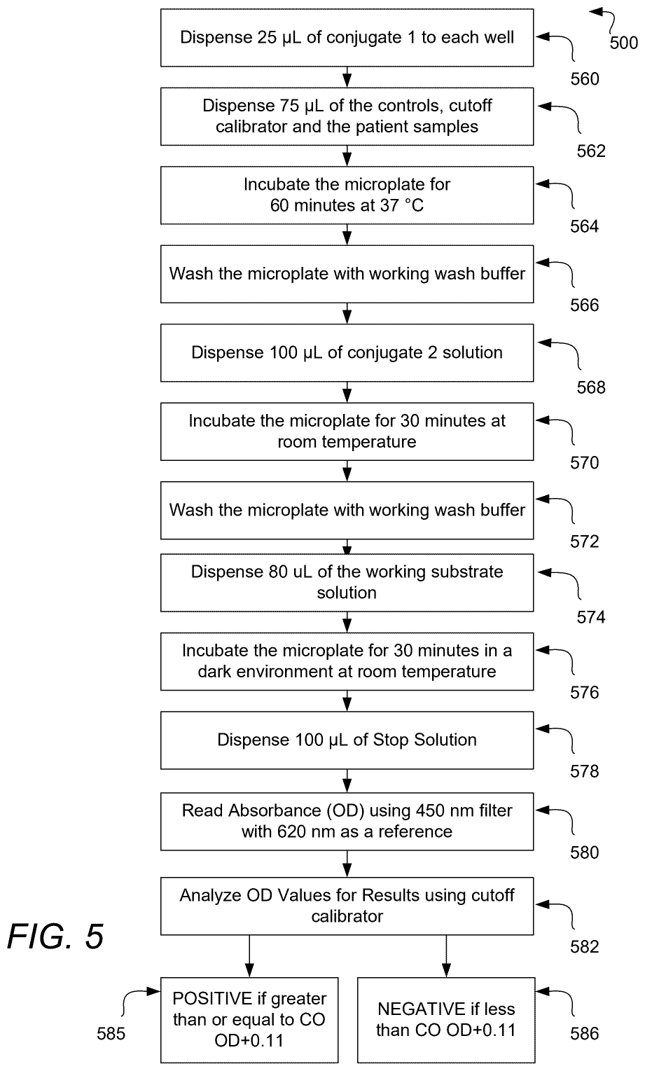

FIGS. 5-8 are method diagrams of specific DBX protocols for preparing an elution for an instrument prepared for an assay to detect the presence of sexually transmitted infections from human dried blood spots collected remotely according to embodiments of the subject matter disclosed herein.

### HIV Assay

FIG. 5 is a method diagram 500 of specific DBX protocols for preparing an elution for an instrument prepared for an HIV assay to detect the presence of HIV from human dried blood spots collected remotely according to embodiments of the subject matter disclosed herein. The GS HIV Combo Ag/Ab EIA is an enzyme immunoassay kit for the simultaneous qualitative detection of Human Immunodeficiency Virus (HIV) p24 antigen and antibodies to the HIV type 1 (HIV-1 Groups M and 0) and HIV type 2 (HIV−2) in human serum or plasma (HIV package insert). By changing the sample type from serum/plasma, which requires the collection of blood by a phlebotomist, to dried blood spot cards, this allows individuals to easily collect samples at home. This product is intended to provide an accessible, simple, at-home HIV screening test to individuals who are at the risk of being exposed to HIV or other STIs.

Acquired immunodeficiency syndrome (AIDS) is caused by an immunodeficiency virus transmitted by sexual contact, exposure to blood or certain blood products (including sharing contaminated needles and syringes) or transmitted from an infected mother to her fetus or child during the perinatal period. Additionally, transmission of these viruses can occur through tissue transplantation (HIV package insert). The majority of people newly infected by the virus develop a flu-like illness within a month or two, having symptoms such as fever, sore throat, headache, muscle aches, swollen lymph glands and so on. Although the symptoms may be mild enough to go unnoticed, the amount of virus in the bloodstream is particularly high. As a result, HIV infection spreads more efficiently during primary infection than during the next stage of infection. Primary infection is followed by a phase of clinical latent infection, which lasts around 10 years without specific signs and symptoms other than swelling of lymph nodes. This phase can last for decades in people taking anti-retroviral medications. Although some people progress to more severe disease much sooner. As the virus continues to multiply and destroy immune cells, mild infections or chronic signs and symptoms will appear, including fever, fatigue, swollen lymph nodes, diarrhea, weight loss, oral yeast infection (thrush), shingles (herpes zoster). By the time AIDS develops, the person's immune system has been severely damaged, making one susceptible to opportunistic infections. The signs and symptoms of some of these infections may include soaking night sweats, recurring fever, chronic diarrhea, persistent white spots or unusual lesions on the tongue or in the mouth, persistent, unexplained fatigue, weight loss, skin rashes or bumps (Mayo Clinic HIV/AIDS symptoms). Both human immunodeficiency virus Type 1 (HIV-1) and human immunodeficiency virus Type 2 (HIV-2) were isolated from patients with AIDS and AIDS-related complex. These two viruses share similarities in morphology, lymphotropism and transmission modes. Their genomes exhibit about 60% homology in conserved genes such as gag and pol and 39%-45% homology in the envelope genes. Serologic studies have also shown that the core proteins of HIV-1 and HIV-2 display frequent cross-reactivity, whereas the envelope proteins are more type-specific (HIV package insert).

Within the 2 major HIV types, significant variation has been found. HIV-1 can be divided into 4 groups: Group M (for major), Group O (for outlier), Group N (for non-M, non-O) and Group P. Similarly, the HIV-2 strains have been classified into at least 5 subtypes (A through E). The GS HIV Combo Ag/Ab EIA incorporates highly conserved recombinant and synthetic peptide sequences representing HIV-1 (Group M and 0) and HIV-2, as well as monoclonal antibodies specific for HIV-1 p24 antigen, which allows for the simultaneous detection of HIV p24 antibody and anti-HIV-1 and anti-HIV-2 antibodies. Because HIV antigens and antibodies appear and are detectable at different stages of seroconversion of the infection, this test can significantly reduce the serological window for detection of HIV (HIV package insert).

The method 500 depicted in FIG. 5 has been validated as discussed below. Prior to discussing validation, the steps of the method are presented. In this method, once an elution of DBS chips is created, Step 560 commences with dispensing 25 μL μL of a first conjugate into each elution well on the microplate. Next, at step 562, a technician may dispense 75 μL of the controls, cutoff calibrator and the patient samples into each well on the microplate. Next, at step 564, the technician may incubate the microplate for 60 minutes at 37° C. followed by washing the microplate with a working wash buffer at step 566. This three-step sub-process may be repeated as step 568 continues with dispensing 100 μL of a second conjugate into each elution well on the microplate. Next, at step 570, the technician may incubate the microplate for 30 minutes at room temperature followed by washing the microplate with a working wash buffer at step 572. This three-step sub-process may be repeated again as step 574 continues with dispensing 80 μL of a working substrate solution into each elution well. Next, at step 576, the technician may incubate the microplate for 30 minutes in a dark environment at room temperature followed by dispensing stop solution at step 578.

After the multiple sets of steps, the microplate may be read. A technician at step 580 may read Absorbance (OD) using 450 nm filter with 620 nm as a reference to analyze OD Values for results using cutoff calibrator at step 582. If the read OD value is greater than or equal to CO OD+0.11, then the determination is a positive result at step 585 (e.g., determine the presence of HIV). However, If the read UD value less than CO OD+0.11, then the determination is a negative result at step 586 (e.g., determine lack of presence of HIV).

This method 500 is validated for precision. Precision, also referred to as reproducibility, is a statistical measure of the variation between repeated test results on the same sample. Three types of variations were analyzed; Intra-run variation, i.e., repeats within the same run; intra-day variation, i.e., between runs, and inter-day variation, i.e., from day to day. The statistics conventionally used to express the precision profile of an assay are the mean (X), standard deviation (SD), and the coefficient of variation (CV); which is calculated by dividing the SD by the mean and multiplying by 100 to express it as a percentage:

$$CV ( \% ) = ( SD / X ) \times 100$$

Seven unique patient samples were selected for validation of the precision of the assay. For each of the seven patient samples, ten contrived DBS cards, were utilized. In order to construct a well-represented precision profile for the HIV EIA, all seven precision samples were run in duplicate for a total of 20 days. All the data of each sample over 20 days was used in the calculation of the intra-run variation and inter-day variation according to Clinical and Laboratory Standards Institute guideline: CLSI EPV5-A2. Samples were run twice on an additional day, with each sample in triplicate on each run. For each sample all 6 data points were used in the calculation of the intra-day variation. In order to demonstrate the reproducibility of the HIV assay, runs were executed by alternating testing personnel at different times of the day, i.e., morning and afternoon runs, for the entire duration of the precision testing period.

In an effort to further validate the test results, one may adjust cutoff and accuracy. The preliminary result of the serological tests is the optical density (OD). However, in order to determine the diagnostic sensitivity and specificity of these assays, the test results must first be qualitatively identified as positive (detected) or negative (undetected). This is achieved by the insertion of a cutoff point on the continuous scale of test results. GS HIV Combo Ag/Ab EIA kits have an established cutoff formula based on thousands of serum/plasma samples. The Cutoff value=Mean absorbance of the Cutoff Calibrator+0.200, where 0.200 is a constant that was predetermined experimentally However, the DBS samples have diluted concentrations of antibodies/antigens compared to standard serum/plasma samples, therefore, the cutoff for DBS samples needed to be re-established. The constant was re-determined based on new data from DBS samples. A total of 751 self-collected DBS cards, including 366 HIV positive samples and 385 HIV negative samples, were tested for estimation of accuracy. A new constant was selected to achieve the best sensitivity and specificity combination.

In an effort to further validate the test results, one may analyze stability. A total of nine samples were utilized to identify the stability of DBSs for the HIV EIA test; three DBS cards were generated from synthetic blood, which yielded low-positive signals, and six DBS cards were generated from patient blood with known high-positive signals. All of the DBS cards were stored at room temperature. In order to yield a signal within the detection range the eluent derived from high-positive blood cards was diluted, ranging from 8-50 times, prior to testing. Tests were run in Week 0 (within three days of the DBS card generation), Week 1, Week 2, Week 4, Week 6, Week 8, and Week 10 (if Week 8 data was not available). All of the OD values were plotted against the time of testing and regression analysis was done using Microsoft Excel. A p-value larger than 0.05 suggests that no significant degradation occurred during the testing period.

In an effort to further validate the test results, one may assess carryover. To determine the possibility of using one puncher to punch multiple separate sample cards, the influence (carryover) of high-positive patient samples to subsequently, punched negative patient samples was tested. One clean puncher was used to punch a known high-positive patient sample 20 times. This same “dirty” puncher was then used to punch a known negative patient sample five times. Each of these five negative punches were collected into five separated test tubes for elution and testing. These five punches were labelled as test punch according to the order of generation as “Test Punch-1” to “Test Punch-5”. A punch of this known positive sample was included as the positive control, while a punch of this known negative sample made by a clean puncher was used as the negative control. Carryover was assessed by determining the differences between the test punches and the clean negative control. In order to statistically calculate the differences, five known positive samples were paired with five known negative samples, and five sets of test punches were made and tested. Student t-test (one tail, type one) was used to analyze the differences.

Interference. Seven potentially interfering substances were identified as commonly encountered substances during the collection of dried blood spot sample material. These 7 substances are outlined in Table 1-1. Each substance was dissolved into elution buffer to a 1% dilution. These solutions were utilized within 18 hours of generation and stored at 4° C. when not in use. Three unique, known-positive samples and three known-negative samples were chosen and eluted utilizing each of the 7 test solutions. Blank elution buffer was used as a negative control.

TABLE 1-1
Overview of potentially interfering
substance for the interference test.

| ID | Sub./Sol. | Description |
|---|---|---|
| A | Ethanol | 70% Ethanol |
| B | Soil | Common soil sample (Vancouver, WA) |
| C | Hair gel | Dep Sport Endurance Styling Gel |
| D | Hand sanitizer | Purell ® Advanced Hand Sanitizer |
| E | Lotion | Jergens ® Extra Dry Skin Moisturizer |
| F | Saliva | Human saliva |
| G | Soap | Softsoap ® Lavender & Chamomile Hand Soap |
| H | N/A (Control) | No treatment - Elution buffer |

Results and Discussion of Precision. All positive samples yielded an Inter-day CV %, an Intra-day CV % and an Intra-run CV % within the acceptable range (<33%), refer to Table 1-2. Negative samples tended to have a larger CV %, with a maximum of 50.3%. However, negative samples did yield lower absolute-OD values; in general, lower values are associated with a large variation. Additionally, all of the EIA results were reported qualitatively, positive call vs. negative call. Of all the negative samples tested none of the results were above the cutoff, which would have hypothetically yielded false low positives. Taking these 2 factors into consideration, it follows that the larger CV % remained acceptable for the HIV assay.

TABLE 1-2
Summary of precision tests for HIV EIA.

| [symbol] OD | | | | [symbol] DayVariation(OD) | | | | [symbol] (OD) | | | |
|---|---|---|---|---|---|---|---|---|---|---|---|
| Sample | Mean | SD | CV % | Sample | Mean | SD | CV % | Sample | Mean | SD | CV % |
| POS1078 | 2.843 | 0.047 | 1.60% | POS1078 | 2.458 | 0.532 | 21.60% | POS1078 | 2.458 | 0.335 | 13.60% |
| POS1085 | 1.143 | 0.074 | 6.50% | POS1085 | 0.95 | 0.208 | 21.90% | POS1085 | 0.95 | 0.143 | 15.00% |
| POS1149 | 2.03 | 0.19 | 9.40% | POS1149 | 1.734 | 0.42 | 24.20% | POS1149 | 1.734 | 0.248 | 14.30% |
| POS1176 | 2.663 | 0.096 | 3.60% | POS1176 | 2.209 | 0.308 | 14.00% | POS1176 | 2.209 | 0.242 | 11.00% |
| STD1266 | 2.759 | 0.142 | 5.10% | STD1266 | 2.213 | 0.442 | 20.00% | STD1266 | 2.213 | 0.262 | 11.80% |
| STD1250 | 0.054 | 0.005 | 8.70% | STD1250 | 0.053 | 0.011 | 21.50% | STD1250 | 0.053 | 0.002 | 3.50% |
| STD1270 | 0.054 | 0.003 | 6.10% | STD1270 | 0.069 | 0.034 | 48.50% | STD1270 | 0.069 | 0.035 | 50.30% |

[symbol]

indicates data missing or illegible when filed

Results and discussion of the DBS Cutoff adjustment and Accuracy Analysis. A total of 751 patient-collected DBS samples were tested; among them, 412 of these were within 8 weeks. The package insert for the HIV EIA defined the cutoff by adding the mean OD of the HIV calibration controls (CCx) to a Constant A.

$$HIV Cutoff ( Package Insert ) = CCx + Constant A .$$

For serum/plasma samples, Constant A was set to 0.200. In order to identify the most ideal cutoff for the DBS samples, various constants were substituted into the cutoff formula. The sensitivity and specificity for the DBS samples were calculated based on the application of the various constants, refer to Table 1-3. According to the ROC curve generated from Table 1-3, the constant that proved to be the most efficacious was 0.110. Therefore, the cutoff formula for DBS samples would be:

$$HIV Cutoff ( DBS Sample ) = CCx + 0.11 .$$

TABLE 1-3
The sensitivity and specificity of the HIV EIA after applying different constants to the cutoff formula.

| | | | | | | | | | | | | | | |
|---|---|---|---|---|---|---|---|---|---|---|---|---|---|---|
| Constant A | 0.00 | 0.05 | 0.10 | 0.11 | 0.12 | 0.15 | 0.16 | 0.17 | 0.18 | 0.19 | 0.20 | a.so | 1.00 | 10.00 |
| Sensitivity | 1.00 | 1.00 | 1.00 | 1.00 | 0.99 | 0.99 | 0.99 | 0.99 | 0.99 | 0.99 | 0.99 | 0.99 | 0.99 | 0.00 |
| Specificity | 0.04 | 0.99 | 1.00 | 1.00 | 1.00 | 1.00 | 1.00 | 1.00 | 1.00 | 1.00 | 1.00 | 1.00 | 1.00 | 1.00 |
| I-Specificity | 0.96 | 0.01 | 0.00 | 0.00 | 0.00 | 0.00 | 0.00 | 0.00 | 0.00 | 0.00 | 0.00 | 0.00 | 0.00 | 0.00 |

Using this cutoff, the assay has a sensitivity of 100% (366/366) and a specificity of 100% (385/385) for all the samples tested. It also resulted in a sensitivity of 100% (268/268) and a specificity of 100% (144/144) for samples tested within eight weeks after generation, refer to Table 1-4.

TABLE 1-4
Comparison of in-house and reference lab HIV test results
for DBS samples that were tested within 8 weeks.

| | MTL | | |
|---|---|---|---|
| Reference | Positive | Negative | Total |
| Positive | 268 | 0 | 268 |
| Negative | 0 | 144 | 144 |
| Total | 268 | 144 | 412 |

Results and discussion of Stability. Nine DBS samples were tracked over a period of 10 weeks. For a complete summary of the stability data refer to Table 1-5. Regression analysis showed that none of the samples exhibited significantly decreased test signals within the allotted time frame. Therefore, it was concluded that for HIV EIA testing, DBS samples are stable for at least eight-week post-generation.

TABLE 1-5
OD values for all DBS samples tested on the HIV stability assay.

| | Samples | | | | | | | | |
|---|---|---|---|---|---|---|---|---|---|
| | PSHS | POS | PSHS | | | | PSHS | PSHS | PSHS |
| | 1046 | 1046 | 1085 | HIV- | HIV- | HIV- | 1148 | 1149 | 1176 |
| Week | 1:8 | 1:8 | 1:8 | 4 | 9 | 24 | 1:50 | 1:50 | 1:50 |
| 0 | 2.722 | 3.054 | 2.410 | 0.483 | 1.817 | 0.190 | 1.760 | 0.959 | 1.860 |
| 1 | 3.069 | 3.217 | 1.630 | 0.507 | 2.598 | 0.148 | 3.100 | 3.100 | 2.079 |
| 2 | 2.598 | 2.736 | 1.475 | 0.513 | 3.028 | 0.130 | 0.793 | 1.491 | 2.782 |
| 4 | 2.369 | 2.677 | NA | 0.791 | 3.168 | 0.136 | 2.497 | 1.514 | 1.400 |
| 6 | 2.202 | 2.914 | 2.278 | 0.469 | 2.886 | 0.112 | 1.142 | 0.830 | NA |
| 8 | 2.324 | 2.985 | 2.517 | 0.371 | 2.433 | 0.120 | NA | NA | 1.537 |
| 10 | NA | NA | NA | NA | NA | NA | 1.500 | 0.842 | NA |
| p-value | 0.053 | 0.631 | 0.381 | 0.668 | 0.550 | 0.057 | 0.560 | 0.316 | 0.393 |

Results and discussion of Carryover. To determine the possibility of using one puncher to punch multiple, separate sample cards, the influence (carryover) of high-positive patient samples to subsequently punched negative patient samples was tested. This experiment revealed that after generating 20 high-positive punches, i.e., punching five unique patient samples four times each, carryover was identified. Negative samples punched after the high positives resulted in an average increase of 14% with regard to the negative sample signal, refer to Table 1-7. While the difference was statistically significant, the increase was not large enough to convert any negative results to a false positive result, refer to Table 1-6.

Additionally, the signal of Test Punch-2 to Test Punch-5 were not significantly different from the negative sample signals, suggesting that the contamination on the puncher can be cleaned by punching once on a clean/blank DBS card.

TABLE 1-6
Average OD values for all DBS samples tested in the carryover experiment.

| Positive | Positive | Negative | Negative | Test | Test | Test | Test | Test |
|---|---|---|---|---|---|---|---|---|
| Sample ID | Card OD | Sample ID | Card OD | Punch-1 | Punch-2 | Punch-3 | Punch-4 | Punch-5 |
| POS1100 | OVER | STD1217 | 0.073 | 0.082 | 0.094 | 0.075 | 0.113 | 0.071 |
| POS1106 | OVER | STD1227 | 0.069 | 0.077 | 0.072 | 0.076 | 0.069 | 0.074 |
| POS1110 | OVER | STD1228 | 0.076 | 0.092 | 0.075 | 0.075 | 0.074 | 0.073 |
| POS1117 | OVER | STD1235 | 0.085 | 0.094 | 0.077 | 0.076 | 0.079 | 0.074 |
| POS1123 | OVER | STD1242 | 0.071 | 0.082 | 0.078 | 0.075 | 0.074 | 0.072 |
| | | | t-test | 0.001 | 0.210 | 0.430 | 0.230 | 0.260 |

TABLE 1-7
Fold change of DBS test samples normalized to known-
negative values (Sample OD/Known-Negative OD).

| Positive | Positive | Negative | Negative | Test | Test | Test | Test | Test |
|---|---|---|---|---|---|---|---|---|
| Sample ID | Card OD | Sample ID | Card OD | Punch-1 | Punch-2 | Punch-3 | Punch-4 | Punch-5 |
| POS1100 | OVER | STD1217 | 1.000 | 1.131 | 1.290 | 1.028 | 1.552 | 0.979 |
| POS1106 | OVER | STD1227 | 1.000 | 1.116 | 1.044 | 1.101 | 0.993 | 1.073 |
| POS1110 | OVER | STD1228 | 1.000 | 1.204 | 0.980 | 0.980 | 0.967 | 0.961 |
| POS1117 | OVER | STD1235 | 1.000 | 1.112 | 0.911 | 0.894 | 0.929 | 0.870 |
| POS1123 | OVER | STD1242 | 1.000 | 1.155 | 1.092 | 1.056 | 1.042 | 1.014 |
| | | | Average | 1.144 | 1.063 | 1.012 | 1.097 | 0.979 |

Results and discussion of Interferences. The sensitivity of the HIV EIA test to interferences was tested. The influence of seven substances that are commonly encountered during DBS sample collection were focused on. Based on the established CV % from the precision study, an SD value was calculated for each sample at its signal level, refer to Table 1-8. The differences between samples containing and those without potential interfering substances were within #3 SD range, refer to Table 1-9. Therefore, it has been concluded that the HIV EIA test is not significantly affected by substances such as 1% of ethanol, soil, hair gel, hand sanitizer, lotion, saliva, and soap products.

TABLE 1-8
OD values for HIV positive and negative samples
tested in the interference experiment.

| Potential | | OD Value (AU) | | | | | |
|---|---|---|---|---|---|---|---|
| Interference | | HIVP1 | HIVP2 | HIVP3 | N1 | N2 | N3 |
| A | Ethanol | 1.172 | OVER | 0.600 | 0.039 | 0.040 | 0.046 |
| B | Soil | 1.228 | OVER | 0.630 | 0.038 | 0.042 | 0.042 |
| C | Hair gel | 0.979 | OVER | 0.656 | 0.043 | 0.042 | 0.046 |
| D | Hand sanitizer | 1.141 | OVER | 0.653 | 0.038 | 0.038 | 0.044 |
| E | Lotion | 1.067 | OVER | 0.612 | 0.036 | 0.038 | 0.041 |
| F | Saliva | 1.007 | OVER | 0.656 | 0.039 | 0.036 | 0.040 |
| G | Soap | 1.008 | OVER | 0.670 | 0.036 | 0.031 | 0.037 |
| H | N/A (Control) | 1.105 | OVER | 0.637 | 0.038 | 0.039 | 0.042 |

| | | | | | | |
|---|---|---|---|---|---|---|
| Precision CV %ₐ | 23.23 | N/A | 23.23 | 20.60 | 20.60 | 20.60 |
| Calculated SD_{b} | 0.257 | N/A | 0.148 | 0.008 | 0.008 | 0.009 |

ₐCV % established from Precision tests
_{b}SD = CV %*Control/100

TABLE 1-9
Significance of potential interfering substances on the HIV test.

| Potential | | Significance | | | | | |
|---|---|---|---|---|---|---|---|
| Interference | | HIVP1 | HIVP2ₐ | HIVP3 | NI | N2 | N3 |
| A | Ethanol | 0 | N/A | 0 | 0 | 0 | 0 |
| B | Soil | 0 | N/A | 0 | 0 | 0 | 0 |
| C | Hair gel | 0 | N/A | 0 | 0 | 0 | 0 |
| D | Hand sanitizer | 0 | N/A | 0 | 0 | 0 | 0 |
| E | Lotion | 0 | N/A | 0 | 0 | 0 | 0 |
| F | Saliva | 0 | N/A | 0 | 0 | 0 | 0 |
| G | Soap | 0 | N/A | 0 | 0 | 0 | 0 |
| H | N/A (Control) | 0 | N/A | 0 | 0 | 0 | 0 |

Note:
“0” 1nd1cates differences between test samples and controls were less than ± 3SO, i.e. not significant. “1” indicates difference were larger than ± 3SD, i.e. significant.
ₐSample value was beyond the detectable range.

In conclusion, the GS HIV Combo Ag/Ab EIA has been successfully validated on the Dynex DSX automated ELISA system with patient self-collected DBS cards (GE Whatman™ 903 protein saver cards) by Molecular Testing Labs and is ready for clinical implementation. This assay achieved 100% sensitivity and specificity based on more than 800 patient self-collected DBS samples. The inter-day precision exhibited a CV less than 33% and the intra-day precision exhibited a CV less than 10%. DBS samples have been shown to be stable for a minimum of 8 weeks post-generation when stored at room temperature. Additionally, no interference was observed from common substances such as 1% ethanol, soil samples, hand sanitizer, lotion, saliva, and soap products.

### HCV Assay

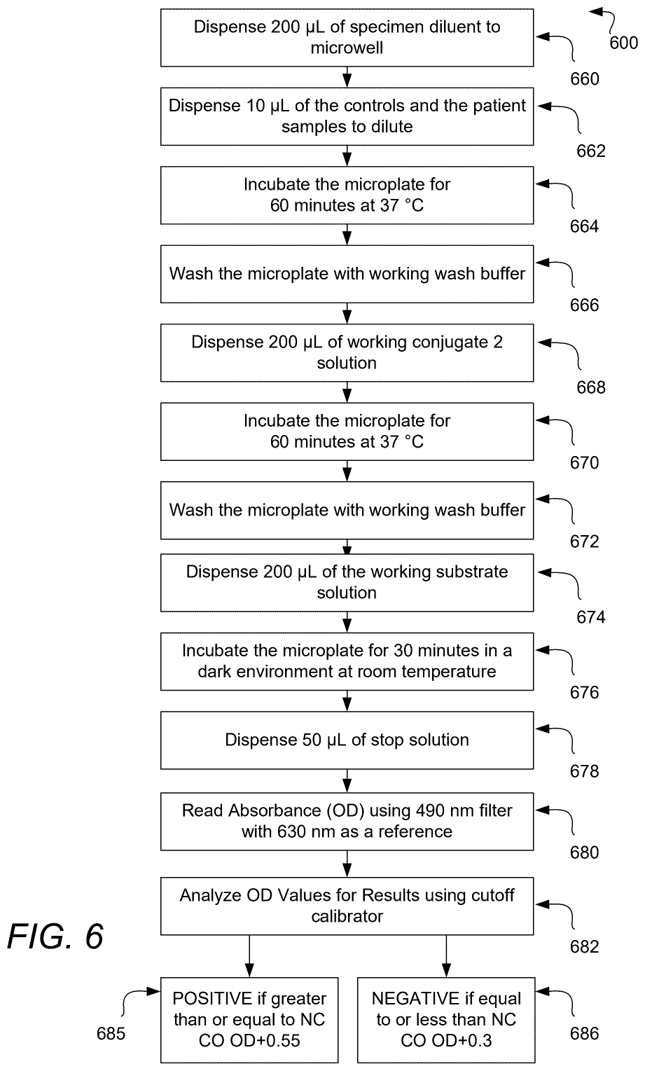

FIG. 6 is a method diagram 600 of specific DBX protocols for preparing an elution for an instrument prepared for an HCV assay to detect the presence of HCV from human dried blood spots collected remotely according to embodiments of the subject matter disclosed herein. The ORTHO® HCV Version 3.0 ELISA Test System is an enzyme-linked, immunosorbent assay for the qualitative detection of antibody to hepatitis C virus (HCV) in human serum and plasma. By changing the sample type from serum/plasma, which requires the collection of blood by a phlebotomist, to dried blood spot cards, this allows individuals to easily collect samples at home. This product is intended to provide an accessible, simple, at-home HCV screening test to individuals who are at the risk of being exposed to STIs.

Hepatitis C is a blood-borne liver disease caused by HCV infection. Six unique HCV genotypes have been identified, all of which can lead to Hepatitis C. Globally 130-185 million people have chronic hepatitis C infections and 2.7 million alone are from the United States. Individuals born during 1945-1965 make up the majority of infected individuals, because it is likely that most infection occurred during the 1970s and/or 1980s (CDC HepC FAQ). Although less infections occur now than in the past HCV is responsible for the deaths of roughly 350,000 people each year due to liver complications/failure (WHO Hepatitis C 2010).

Hepatitis C can be either “acute” or “chronic.” Acute Hepatitis C virus infection is a short-term illness that occurs within the first six months after someone is exposed to the Hepatitis C virus. Symptoms may include fever, fatigue, dark urine, clay-colored stool, abdominal pain, loss of appetite, nausea, vomiting, joint pain, and/or jaundice (CDC HepC FAQ). When symptoms are exhibited, they are typically mild and unlikely to prompt an individual to seek medical attention. Roughly 80% of initially infected individuals are asymptomatic and may be unaware of their infection status, which in turn perpetuates the virus. Through unknown mechanisms, approximately 15%-25% of infected persons clear the virus from their bodies without treatment and do not develop chronic infection. The rest 70%-85% of infected people who do not spontaneously clear the virus will develop a chronic hepatitis C infection (WHO Hepatitis C 2010). Chronic Hepatitis C is a serious disease than can result in serious health complications; chronic liver disease, cirrhosis, liver cancer, general liver damage (e.g., fibrosis}, co-infection/susceptibility to other hepatitis viruses (HAVand HBV), all of which can lead to death.

The most common means of HCV transmission occurs through injections with infected needles (via inadequate sterilization of equipment in health care settings or with “dirty” needles for illicit drug use), unscreened/infected organ and blood transfusions. Although less common the HCV can be transmitted through sexual contact with infected individuals, and transmission from mother to child during birth. Populations considered “high-risk” for HCV infection include illicit drug users (via injection and intranasal), recipient of tattoos/piercings, individuals in prison or previously incarcerated, individuals infected with HIV, individuals with HCV-infected sexual partners, chronic hemodialysis patients, and recipients of infected/inadequately sterilized healthcare products (CDC HepC FAQ).

There is no vaccination currently available for HCV, therefore, the best means of prevention is through education of the disease, rigorous sterilization/screening requirements in health care settings, avoidance of high-risk behaviors, protected sex, and accessibility to screening tests for the general public. Follow-up testing is recommended for individuals who test positive for HCV on screening tests. Targeted nucleic acid testing for HCV RNA is a common means of confirmatory testing, additionally a liver biopsy to assess liver damage is recommended concurrently (IDSA HCV Guidance).

The standard treatment of chronic hepatitis C is rapidly changing, but a common means is the use of antiviral drugs. Recently a new set of medications targeting HCV have been developed, these are known as direct-acting antivirals (DAAs). Adherence to 12 weeks of treatment with DAAs has yielded positive results in clinical studies; an absence of the virus was noted even after the completion of the treatment.

The method 600 depicted in FIG. 6 has been validated as discussed below. Prior to discussing validation, the steps of the method are presented. In this method, once an elution of DBS chips is created, Step 660 commences with dispensing 200 μL of specimen diluent to a microwell on the microplate. Next, at step 662, a technician may dispense 10 μL of the controls and the patient samples into the microwell on the microplate. Next, at step 664, the technician may incubate the microplate for 60 minutes at 37° C. followed by washing the microplate with a working wash buffer at step 666. This three-step sub-process may be repeated as step 668 continues with dispensing 200 μL of a second conjugate into each the microwell on the microplate. Next, at step 670, the technician may incubate the microplate for 60 minutes at 37° C. followed by washing the microplate with a working wash buffer at step 672. This three-step sub-process may be repeated again as step 674 continues with dispensing 200 μL of a working substrate solution into each elution well. Next, at step 676, the technician may incubate the microplate for 30 minutes in a dark environment at room temperature, however, without following up with any washing.

After the multiple sets of steps, the microplate may be read after step 678 whereby 50 mL of stop solution are disponed into the microwell of the microplate. A technician at step 680 may read Absorbance (OD) using 490 nm filter with 630 nm as a reference to analyze Optical Density (OD) Values for results using cutoff calibrator at step 682. If the read OD value is greater than or equal to NC CO OD+0.55, then the determination is a positive result at step 685 (e.g., determine the presence of HCV). However, If the read OD value less than CO OD+0.11, then the determination is a negative result at step 686 (e.g., determine lack of presence of HCV).

This method 600 is validated for precision (also referred to as reproducibility) and is a statistical measure of the variation between repeated test results on the same sample. Three types of variations were analyzed; Intra-run variation, i.e., repeats within the same run; intra-day variation, i.e., between runs, and inter-day variation, i.e., from day to day. The statistics conventionally used to express the precision profile of an assay are the mean (X), standard deviation (SD), and the coefficient of variation (CV); which is calculated by dividing the SD by the mean and multiplying by 100 to express it as a percentage.

$$CV ( \% ) = ( SD / X ) \times 100$$

In this experiment, eight unique patient samples were selected for validation of the precision of the HCV assay. For each of the eight patient samples, ten DBS cards generated in-house were utilized. In order to construct a well-represented precision profile for the HIV EIA, all eight precision samples were run in duplicate for a total of 20 days. All the data of each sample over 20 days was used in the calculation of the intra-run variation and inter-day variation according to Clinical and Laboratory Standards Institute guideline. CLSI EPV5-A2. Samples were run twice on an additional day, with each sample in triplicate on each run. For each sample all six data points were used in the calculation of the intra-day variation. In order to demonstrate the reproducibility of the HCV assay, runs were executed by alternating testing personnel at different times of the day, i.e., morning and afternoon runs, for the entire duration of the precision testing period.

Adjusting the Cutoff and Accuracy—The preliminary result of the serological tests is the optical density (OD). However, in order to determine the diagnostic sensitivity and specificity of these assays, the test results must first be qualitatively identified as positive (detected) or negative (not detected). This is achieved by the insertion of a cutoff point on the continuous scale of test results. The commercially available ORTHO® HCV Version 3.0 ELISA Test System has an established cutoff formula based on thousands of serum/plasma samples. It is expressed as Cutoff Value=Mean absorbance of the Negative control+0.600, where 0.600 is a pre-determined constant.

However, DBS samples have diluted concentrations of antibodies/antigens compared to standard serum/plasma samples, therefore, the cutoff for DBS samples needed to be re-established. The constant was re-determined based on new data from DBS samples. A total of 793 self-collected DBS cards, consisting of 244 HCV positive samples and 549 HCV negative samples, were tested for estimation of accuracy. A new constant was selected to achieve the best sensitivity and specificity combination.

Stability—A total of six samples were utilized to identify the stability of DBSs for the HCV assay; all samples were generated from patient blood with known positive signals. All of the cards were stored at room temperature. Tests were run in Week 0 (within three days of the DBS card generation), Week 1, Week 2, Week 4, Week 6, and Week 8. All of the OD values were plotted against the time of testing and regression analysis was done using Microsoft Excel. A p-value larger than 0.05 suggests that no significant degradation occurred during the testing period.

Carryover—To determine the possibility of using one puncher to punch multiple, separate sample cards, the influence (carryover) of high-positive patient samples to subsequently punched negative patient samples was tested. One clean puncher was used to punch a known high-positive patient sample 20 times. This same “dirty” puncher was then used to punch a known negative patient sample five times. Each of these five negative punches were collected into five separated test tubes for elution and testing. These five punches were labelled as test punch according to the order of generation as “Test Punch-1” to “Test Punch-5”. A punch of this known positive sample was included as the positive control, while a punch of this known negative sample made by a clean puncher was used as the negative control. Carryover was assessed by determining the differences between the test punches and the clean negative control. In order to statistically calculate the differences, five known positive samples were paired with five known negative samples, and five sets of test punches were made and tested. Student t-test (one tail, type one) was used to analyze the differences.

Interference—Seven potential interfering substances were identified as commonly encountered substances during the collection of dried blood spot sample material. These seven substances are outlined in Table 2-1. Each substance was dissolved into elution buffer (e.g., in one embodiment, PBS+0.05% TWEEN-20) to a 1% dilution. These solutions were utilized within 18 hours of generation and stored at 4° C. when not in use. Three unique, known-positive samples and three known-negative samples were chosen and eluted utilizing each of the seven test solutions. Blank elution buffer was used as a negative control.

TABLE 2-1
Overview of potential interfering substance
for the interference test.

| ID | Sub./Sol. | Description |
|---|---|---|
| A | Ethanol | 70% Ethanol |
| B | Soil | Common soil sample (Vancouver, WA) |
| C | Hair gel | Dep Sport Endurance Styling Gel |
| D | Hand sanitizer | Purell ® Advanced Hand Sanitizer |
| E | Lotion | Jergens ® Extra Dry Skin Moisturizer |
| F | Saliva | Human saliva |
| G | Soap | Softsoap ® Lavender & Chamomile Hand Soap |
| H | N/A (Control) | No treatment - Elution buffer |

Cross-reactivity of HCV assay to HIV, HSV-2 and Syphilis. In addition to testing potential interferences coming from commonly encountered substances, we also determined if the HCV assay cross-reacted with any of the other three disease types tested, such as HIV, Syphilis and HSV-2. A total of 801 samples that were tested on all four assays was used in this analysis. For instance, when cross-reactivity of the HCV EIA to HIV EIA signals was analyzed, all Syphilis-/HSV-2-samples were separated into four groups: HIV−/HCV−, HIV+/HCV−, HIV−/HCV+, and HIV+/HCV+. In each group, samples were ranked from the lowest OD value to the highest OD value. Analysis bins were then generated based on HCV OD value for a total range from 0.0 to 3.5. The frequency of samples that fell into each analysis bin was calculated. The frequencies were plotted again analysis bin values to allow easy visualization of sample distributions in each of the groupings. If HIV+ disease type samples were to affect the HCV test signals, the distribution of HIV+/HCV− and HIV−/HCV− would be clearly different. The same methodology was used to analyze the potential cross-reactivity of the HCV assay to other disease types.

Results and discussion for Precision. All positive samples yielded an Inter-day CV %, an Intra-day CV % and an Intra-run CV % within the acceptable range (<33%). Refer to Table 2.2. Negative samples tended to have a larger CV %, with a maximum of 47.46%. However, negative samples did yield lower absolute-OD values; in general, lower values are associated with a large variation. Additionally, all of the EIA results were reported qualitatively, positive call vs. negative call. Of all the negative samples tested none of the results were above the cutoff, which would have hypothetically yielded false low positives. Taking these two factors into consideration it follows that the larger CV percentages remain acceptable for the HIV assay.

TABLE 2-2
Summary of precision tests for HCV EIA.

| Intra-Day Variatioh(6fepe;!c1ts) OD | | | | Intef-r,ay Variation (OD) | | | | Intra Run Variation(OD) | | | |
|---|---|---|---|---|---|---|---|---|---|---|---|
| Sample | Mean | SD | CV % | Sample | Mean | SD | CV % | Sample | Mean | SD | CV % |
| POS1065 | 0.354 | 0.054 | 15.32% | POS1065 | 0.532 | 0.126 | 23.69% | POS1065 | 0.532 | 0.05 | 9.36% |
| POS1073 | 0.617 | 0.075 | 12.07% | POS1073 | 0.714 | 0.152 | 21.27% | POS1073 | 0.714 | 0.07 | 9.84% |
| POS1082 | 0.357 | 0.062 | 17.44% | POS1082 | 0.473 | 0.101 | 21.32% | POS1082 | 0.473 | 0.083 | 17.57% |
| POS1096 | 2.890 | 0.037 | 1.27% | POS1096 | 2.885 | 0.053 | 1.85% | POS1096 | 2.885 | 0.032 | 1.09% |
| POS1097 | 2.910 | 0.051 | 1.75% | POS1097 | 2.903 | 0.047 | 1.63% | POS1097 | 2.903 | 0.041 | 1.40% |
| POS1098 | 2.934 | 0.036 | 1.23% | POS1098 | 2.903 | 0.046 | 1.58% | POS1098 | 2.903 | 0.047 | 1.63% |
| STD1261 | 0.042 | 0.007 | 17.13% | STD1261 | 0.084 | 0.04 | 47.46% | STD1261 | 0.084 | 0.019 | 22.83% |
| STD1285 | 0.061 | 0.025 | 41.93% | STD1285 | 0.122 | 0.05 | 41.44% | STD1285 | 0.122 | 0.022 | 17.95% |

Results and discussion for Adjustment of the DBS Cutoff and Accuracy. A total of 793 patient-collected DBS samples were tested to adjust the DBS cutoffs. Refer to Table 2.3. 244 of these were positive and 549 of these were negative according to the reference results, i.e., Quest Diagnostics. The package insert for the HCV EIA defined the cutoff by adding the mean OD of the HCV Negative controls (NCx) to a Constant A.

$$HCV cutoff ( Package Insert ) = NCx + Constant A .$$

For serum/plasma samples, Constant A was set to 0.600. In order to identify the most ideal cutoff for the DBS samples various constants were substituted into the cutoff formula. Initial analysis revealed that the best combination of sensitivity and specificity could be achieved when constant A was set at 0.25 (data not shown). However, some discordant samples were identified; these samples, which initially tested positive yielded a negative call during the retests. The reason for the presence of discordant samples is that the sample OD values varied around the cutoff value. This indicates that a single cutoff strategy does not work for DBS samples on the HCV assay. To overcome this problem, an equivocal zone was introduced into the assay, consisting of an upper-cutoff and a lower-cutoff. The formula was re-defined as:

$$HCV upper - cutoff = NCx + upper - cutoff constant \\ HCV lower cutoff = NCx + lower cutoff constant$$

All the samples that have an OD value above the upper cutoff are considered positive and all samples below the lower cutoff are considered negative. Samples in between the upper and lower cutoffs are reported as equivocal. The sensitivity and specificity for the DBS samples were further calculated based on the application of various constants.

From the stability test, it was determined that the DBS samples are stable for eight weeks at room temperature (refer to 2.4.3 for details); therefore, cutoffs were calculated separately for samples tested within 8 weeks vs. all samples. Additionally, it was noted that the HCV positive signal level was different between HIV+ patients and HIV− patients. Therefore, HIV− samples were also separated from the total samples. Overall, there were four distinct scenarios in which the sensitivity and specificity of potential cutoffs were calculated: 1) all samples tested (Table 2-3, n=793); 2) all samples tested within eight weeks of generation (Table 2-4, n=412); 3) all HIV− samples tested (Table 2-5, n=374); and 4) and HIV− samples tested within eight weeks of generation (Table 2-6, n=107). For each scenario, the sensitivity, specificity, and % equivocal for the DBS samples was calculated based on the application of various upper-cutoff and lower-cutoff values.

Because the HIV+ rate is very low in the general population, it is best to use the data from all HIV− samples tested within eight weeks after generation in order to calculate the new cutoffs. As shown in Table 2-6, when the upper-cutoff was set at 0.55 and the lower-cutoff was set at 0.30, the sensitivity and specificity of this data set for the HCV assay were 97.22% and 100.00% respectively. Roughly 6.54% of the samples fell into the equivocal zone. These cutoffs also yielded the best sensitivity/specificity/% equivocal combination in all samples that were less than 8 weeks old. The sensitivity and specificity of for this data set is 95.58% and 98.92% for the HCV assay. Roughly 6.54% of the samples fell into the equivocal zone (Table 2-6). These cutoffs also yielded the best sensitivity/specificity/% equivocal combination in all samples that were less than 8 weeks old. The sensitivity and specificity of for this data set is 95.58% and 98.92% for the HCV assay. Roughly 5.34% samples fell into the equivocal zone (Table 2-4 and Table 2-7). For the other 2 data sets, the cutoffs of 0.30 and 0.55 again achieved the best sensitivity/specificity/% equivocal combination. Therefore, it was determined to use 0.55 as the upper-cutoff constant and 0.30 as the lower cutoff constant for the DBS HCV test.

TABLE 2-3
The statistical measures for various upper-cutoffs and lower-
cutoffs for all samples tested on the HCV EIA (n = 793).

| Lower | [symbol] | | | | | | | | | | | Performance |
|---|---|---|---|---|---|---|---|---|---|---|---|---|
| Cutoff | 0.25 | [symbol] | b.as | 0.40 | 0.45 | 0.50 | [symbol] | 0.60 | 0.65 | 0.70 | 0.75 | Characteristics |
| 0.20 | 98.35 | 98.33 | 98.32 | 98.31 | 98.31 | 98.30 | 98.29 | 98.28 | 98.27 | 98.27 | 98.27 | Sensitivity % |
| | 91.54 | 94.93 | 97.01 | 97.79 | 98.19 | 98.78 | 99.39 | 99.39 | 99.39 | 99.39 | 99.59 | Specificity % |
| | 2.40 | 5.04 | 6.68 | 7.44 | 7.69 | 8.20 | 8.70 | 8.95 | 9.08 | 9.08 | 9.21 | % Equivocal |
| 0.25 | 97.54 | 97.52 | 97.50 | 97.48 | 97.48 | 97.47 | 97.46 | 97.44 | 97.42 | 97.42 | 97.42 | Sensitivity % |
| | 91.80 | 95.09 | 97.11 | 97.86 | 98.25 | 98.82 | 99.41 | 99.41 | 99.41 | 99.41 | 99.60 | Specificity % |
| | 0.00 | 2.65 | 4.29 | 5.04 | 5.30 | 5.80 | 6.31 | 6.56 | 6.68 | 6.68 | 6.81 | % Equivocal |
| 0.30 | | 96.72 | 96.69 | 96.67 | 96.67 | 96.65 | 96.64 | 96.61 | 96.60 | 96.60 | 96.60 | Sensitivity % |
| | | 95.26 | 97.21 | 97.94 | 98.31 | 98.87 | 99.43 | 99.43 | 99.43 | 99.43 | 99.62 | Specificity % |
| | | 0.00 | 1.64 | 2.40 | 2.65 | 3.15 | 3.66 | 3.91 | 4.04 | 4.04 | 4.16 | % Equivocal |
| 0.35 | | | 95.90 | 95.87 | 95.87 | 95.85 | 95.83 | 95.80 | 95.78 | 95.78 | 95.78 | Sensitivity % |
| | | | 97.27 | 97.98 | 98.34 | 98.89 | 99.44 | 99.44 | 99.44 | 99.44 | 99.63 | Specificity % |
| | | | 0.00 | 0.76 | 1.01 | 1.51 | 2.02 | 2.27 | 2.40 | 2.40 | 2.52 | % Equivocal |
| 0.40 | | | | 95.08 | 95.08 | 95.06 | 95.04 | 95.00 | 94.98 | 94.98 | 94.98 | Sensitivity % |
| | | | | 98.00 | 98.35 | 98.90 | 99.45 | 99.45 | 99.45 | 99.45 | 99.63 | Specificity % |
| | | | | 0.00 | 0.25 | 0.76 | 1.26 | 1.51 | 1.64 | 1.64 | 1.77 | % Equivocal |

[symbol]

indicates data missing or illegible when filed

TABLE 2-4
The statistical measures for various upper-cutoffs and lower-cutoffs for
all samples tested on the HCV EIA within 8 weeks of generation (n = 412).

| Lower | [symbol] Uppe r-cutoff [symbol] | | | | | | | | | | | Performance |
|---|---|---|---|---|---|---|---|---|---|---|---|---|
| Cutoff | 0.25 | 0.0 | 0.35 | 0.40 | 0.45 | 0.50 | 0.55 | 0.60 | 0.65 | 0.70 | 0.75 | Characteristics |
| 0.20 | 98.29 | 98.26 | 98.23 | 98.21 | 98.21 | 98.20 | 98.18 | 98.15 | 98.15 | 98.15 | 98.15 | Sensitivity % |
| | 89.05 | 92.65 | 95.82 | 96.55 | 96.92 | 98.05 | 98.82 | 98.82 | 98.82 | 98.82 | 99.21 | Specificity % |
| | 2.91 | 6.07 | 8.74 | 9.47 | 9.71 | 10.68 | 11.41 | 11.89 | 11.89 | 11.89 | 12.14 | % Equivocal |
| 0.25 | 97.46 | 97.41 | 97.37 | 97.35 | 97.35 | 97.32 | 97.30 | 97.25 | 97.25 | 97.25 | 97.25 | Sensitivity % |
| | 89.46 | 92.93 | 95.99 | 96.69 | 97.05 | 98.13 | 98.87 | 98.87 | 98.87 | 98.87 | 99.25 | Specificity % |
| | 0.00 | 3.16 | 5.83 | 6.55 | 6.80 | 7.77 | 8.50 | 8.98 | 8.98 | 8.98 | 9.22 | % Equivocal |
| [symbol] | | 95.76 | 95.69 | 95.65 | 95.65 | 95.61 | 95.58 | 95.50 | 95.50 | 95.50 | 95.50 | Sensitivity % |
| 0.30 | | 93.20 | 96.14 | 96.82 | 97.16 | 98.21 | 98.92 | 98.92 | 98.92 | 98.92 | 99.28 | Specificity % |
| | | 0.00 | 2.67 | 3.40 | 3.64 | 4.61 | 5.34 | 5.83 | 5.83 | 5.83 | 6.07 | % Equivocal |
| 0.35 | | | 94.07 | 94.02 | 94.02 | 93.97 | 93.91 | 93.81 | 93.81 | 93.81 | 93.81 | Sensitivity % |
| | | | 96.26 | 96.92 | 97.25 | 98.26 | 98.95 | 98.95 | 98.95 | 98.95 | 99.30 | Specificity % |
| | | | 0.00 | 0.73 | 0.97 | 1.94 | 2.67 | 3.16 | 3.16 | 3.16 | 3.40 | % Equivocal |
| [symbol] | | | | 93.22 | 93.22 | 93.16 | 93.10 | 92.98 | 92.98 | 92.98 | 92.98 | Sensitivity % |
| | | | | 96.94 | 97.27 | 98.28 | 98.96 | 98.96 | 98.96 | 98.96 | 99.30 | Specificity % |
| | | | | 0.00 | 0.24 | 1.21 | 1.94 | 2.43 | 2.43 | 2.43 | 2.67 | % Equivocal |

[symbol]

indicates data missing or illegible when filed

TABLE 2-5
The statistical measures for various upper-cutoffs and lower-
cutoffs for all HIV-samples tested on the HCV EIA (n = 374).

| Lower | [symbol] | | | | | | | | | | | Performance |
|---|---|---|---|---|---|---|---|---|---|---|---|---|
| Ctifoff | 0.25 | 0.30 | 0.35 | 0.4Q | 0.45 [symbol] | 0.50 | 0.55 | 0.60 [symbol] | 0.65 | 0.70 | 0.75 | Characteristics |
| [symbol] | 98.43 | 98.43 | 98.40 | 98.39 | 98.39 | 98.37 | 98.37 | 98.37 | 98.36 | 98.36 | 98.36 | Sensitivity % |
| | 96.31 | 97.11 | 98.33 | 99.16 | 99.16 | 99.16 | 100.00 | 100.00 | 100.00 | 100.00 | 100.00 | Specificity % |
| | 0.80 | 1.34 | 2.67 | 3.48 | 3.48 | 3.74 | 4.28 | 4.28 | 4.55 | 4.55 | 4.55 | % Equivocal |
| [symbol] | 98.43 | 98.43 | 98.40 | 98.39 | 98.39 | 98.37 | 98.37 | 98.37 | 98.36 | 98.36 | 98.36 | Sensitivity % |
| | 96.36 | 97.14 | 98.35 | 99.17 | 99.17 | 99.17 | 100.00 | 100.00 | 100.00 | 100.00 | 100.00 | Specificity % |
| | 0.00 | 0.53 | 1.87 | 2.67 | 2.67 | 2.94 | 3.48 | 3.48 | 3.74 | 3.74 | 3.74 | % Equivocal |
| 0.30 | | 98.43 | 98.40 | 98.39 | 98.39 | 98.37 | 98.37 | 98.37 | 98.36 | 98.36 | 98.36 | Sensitivity % |
| | | 97.17 | 98.36 | 99.17 | 99.17 | 99.17 | 100.00 | 100.00 | 100.00 | 100.00 | 100.00 | Specificity % |
| | | 0.00 | 1.34 | 2.14 | 2.14 | 2.41 | 2.94 | 2.94 | 3.21 | 3.21 | 3.21 | % Equivocal |
| 0.35 | | | 96.85 | 96.83 | 96.83 | 96.80 | 96.80 | 96.80 | 96.77 | 96.77 | 96.77 | Sensitivity % |
| | | | 98.38 | 99.18 | 99.18 | 99.18 | 100.00 | 100.00 | 100.00 | 100.00 | 100.00 | Specificity % |
| | | | 0.00 | 0.80 | 0.80 | 1.07 | 1.60 | 1.60 | 1.87 | 1.87 | 1.87 | % Equivocal |
| 0.40 | | | | 96.06 | 96.06 | 96.03 | 96.03 | 96.03 | 96.00 | 96.00 | 96.00 | Sensitivity % |
| [symbol] | | | | 99.19 | 99.19 | 99.19 | 100.00 | 100.00 | 100.00 | 100.00 | 100.00 | Specificity % |
| | | | | 0.00 | 0.00 | 0.27 | 0.80 | 0.80 | 1.07 | 1.07 | 1.07 | % Equivocal |

[symbol]

indicates data missing or illegible when filed

TABLE 2-6
The statistical measures for various upper-cutoffs and lower-cutoffs for all
HIV-samples tested on the HCV EIA within 8 weeks of generation (n = 107).

| Lower | Upper cutoff | | | | | | | | | | | Performance |
|---|---|---|---|---|---|---|---|---|---|---|---|---|
| Cutoff | 0.25 | 0.30 | 0.35 | 0.40 | 0.45 | 0.50 | 0.55 | 0.60 | 0.65 | 0.70 | 0.75 | Characteristics |
| 0.20 | 97.44 | 97.44 | 97.30 | 97.30 | 97.30 | 97.22 | 97.22 | 97.22 | 97.22 | 97.22 | 97.22 | Sensitivity % |
| | 94.03 | 94.03 | 98.44 | 98.44 | 98.44 | 98.44 | 100.00 | 100.00 | 100.00 | 100.00 | 100.00 | Specificity |
| | 0.93 | 0.93 | 5.61 | 5.61 | 5.61 | 6.54 | 7.48 | 7.48 | 7.48 | 7.48 | 7.48 | % Equivocal |
| 0.25 | 97.44 | 97.44 | 97.30 | 97.30 | 97.30 | 97.22 | 97.22 | 97.22 | 97.22 | 97.22 | 97.22 | Sensitivity % |
| | 94.12 | 94.12 | 98.46 | 98.46 | 98.46 | 98.46 | 100.00 | 100.00 | 100.00 | 100.00 | 100.00 | Specificity % |
| | 0.00 | 0.00 | 4.67 | 4.67 | 4.67 | 5.61 | 6.54 | 6.54 | 6.54 | 6.54 | 6.54 | % Equivocal |
| 0.30 | | 97.44 | 97.30 | 97.30 | 97.30 | 97.22 | 97.22 | 97.22 | 97.22 | 97.22 | 97.22 | Sensitivity % |
| | | 94.12 | 98.46 | 98.46 | 98.46 | 98.46 | 100.00 | 100.00 | 100.00 | 100.00 | 100.00 | Specificity % |
| | | 0.00 | 4.67 | 4.67 | 4.67 | 5.61 | 6.54 | 6.54 | 6.54 | 6.54 | 6.54 | % Equivocal |
| 0.35 | | | 92.31 | 92.31 | 92.31 | 92.11 | 92.11 | 92.11 | 92.11 | 92.11 | 92.11 | Sensitivity % |
| | | | 98.53 | 98.53 | 98.53 | 98.53 | 100.00 | 100.00 | 100.00 | 100.00 | 100.00 | Specificity % |
| | | | 0.00 | 0.00 | 0.00 | 0.93 | 1.87 | 1.87 | 1.87 | 1.87 | 1.87 | % Equivocal |
| 0.40 | | | | 92.31 | 92.31 | 92.11 | 92.11 | 92.11 | 92.11 | 92.11 | 92.11 | Sensitivity % |
| | | | | 98.53 | 98.53 | 98.53 | 100.00 | 100.00 | 100.00 | 100.00 | 100.00 | Specificity % |
| | | | | 0.00 | 0.00 | 0.93 | 1.87 | 1.87 | 1.87 | 1.87 | 1.87 | % Equivocal |

TABLE 2-7
Comparison of in-house and reference lab HCV test results
for DBS samples, using all samples less than 8 weeks old.

| | Reference | | |
|---|---|---|---|
| MTL | Positive | Negative | Total |
| Positive | 108 | 3 | 111 |
| Negative | 5 | 274 | 279 |
| Equivocal | 5 | 17 | 22 |
| Total | 118 | 294 | 412 |

Results and discussion for Stability. Six DBS samples were tracked over a period of eight weeks. For a complete summary of the stability data refer to Table 2-8. Regression analysis showed that none of the samples exhibited significantly decreased test signals within the allotted time frame. Therefore, it was concluded that for HCV EIA testing, DBS samples are stable for at least 8-week post-generation.

TABLE 2-8
OD values for all DBS samples tested on the HCV assay for stability purposes.

| | Samples | | | | | |
|---|---|---|---|---|---|---|
| Week | PSHS1082 | POS1082 | POS1168 | POS1169 | POS1170 | POS1171 |
| 0 | 1.080 | 1.073 | 2.943 | 2.855 | 2.859 | 2.864 |
| 1 | 1.198 | 1.063 | 2.849 | 2.311 | 2.885 | 2.838 |
| 2 | 1.020 | 1.164 | 3.100 | 3.100 | 2.918 | 2.907 |
| 4 | 0.933 | 1.230 | 2.907 | 2.384 | 2.891 | 2.885 |
| 6 | 1.445 | 1.155 | 2.648 | 2.744 | 2.709 | 2.850 |
| 8 | 1.037 | 2.516 | OVER | 1.832 | 2.954 | 2.964 |
| p-value | 0.766 | 0.077 | 0.253 | 0.219 | 0.903 | 0.208 |

To determine the possibility of using one puncher to punch multiple, separate sample cards, the influence (carryover) of high-positive patient samples to subsequently punched negative patient samples was tested. This experiment revealed that after generating 20 high-positive punches, i.e., punching five unique patient samples four times each, a small amount of carryover was identified (Table 2-10). However, the difference was not statistically significant (Table 2-9).

TABLE 2-9
Average OD values for all DBS samples tested on
the HCV EIA test in the carryover experiment.

| Positive | Positive | Negative | Negative | Test | Test | Test | Test | Test |
|---|---|---|---|---|---|---|---|---|
| Sample ID | Card OD | Sample ID | Card OD | Punch-1 | Punch-2 | Punch-3 | Punch-4 | Punch-6 |
| POS1086 | 2.851 | STD1070 | 0.172 | 0.142 | 0.096 | 0.138 | 0.090 | 0.088 |
| POS1094 | 2.858 | STD1081 | 0.068 | 0.081 | 0.088 | 0.073 | 0.089 | 0.079 |
| POS1102 | OVER | STD1082 | 0.083 | 0.092 | 0.085 | 0.104 | 0.071 | 0.082 |
| POS1103 | 2.870 | STD1086 | 0.081 | 0.136 | 0.109 | 0.091 | 0.175 | 0.093 |
| STD1269 | 2.932 | STD1115 | 0.386 | 0.539 | 0.525 | 0.341 | 0.516 | 0.394 |
| | | | t-test | 0.135 | 0.273 | 0.276 | 0.234 | 0.294 |

TABLE 2-10
DBS test samples normalized to known-negative
values (Sample OD/Known-Negative OD).

| Positive | Positive | Negative | Negative | Test | Test | Test | Test | Test |
|---|---|---|---|---|---|---|---|---|
| Sample ID | Card OD | Sample ID | Card OD | Punch-1 | Punch-2 | Punch-3 | Punch-4 | Punch-5 |
| POS1086 | 2.851 | STD1070 | 1.00 | 0.828 | 0.560 | 0.802 | 0.322 | 0.513 |
| POS1094 | 2.858 | STD1081 | 1.00 | 1.184 | 1.287 | 1.074 | 1.309 | 1.154 |
| POS1102 | OVER | STD1082 | 1.00 | 1.109 | 1.024 | 1.261 | 0.855 | 0.988 |
| POS1103 | 2.870 | STD1086 | 1.00 | 1.689 | 1.348 | 1.124 | 2.168 | 1.149 |
| STD1269 | 2.932 | STD1115 | 1.00 | 1.398 | 1.362 | 0.885 | 1.339 | 1.021 |
| | | | Average | 1.242 | 1.116 | 1.029 | 1.238 | 0.965 |

Results and discussion for Interferences. The sensitivity of the HCV EIA test to interferences was tested. The influence of 7 substances that are commonly encountered during DBS sample collection were focused on. Based on the established CV % from the precision study, an SD value was calculated for each sample at its signal level, refer to Table 2-11. If the differences between samples containing potential interfering substances versus those without were larger than ±3 SD range, they were assigned a significance score “1”, otherwise samples were assigned a “0”. As shown in Table 2-12, 1% of ethanol, soil, hair gel, hand sanitizer, lotion can increase the OD value, therefore interfere with the HCV EIA test. The remaining substances: 1% saliva and 1% soap, did not affect the overall signal of HCV.

TABLE 2-11
OD values for HCV positive and negative samples tested utilizing
elution buffers with various potential interfering substances.

| Potential | | OD Value (AU) | | | | | |
|---|---|---|---|---|---|---|---|
| Interference | | HCVP1 | HCVP2 | HCVP3 | N1 | N2 | N3 |
| A | Ethanol | ovERa | 0.360 | 2.881 | 0.168 | 0.158 | 0.110 |
| B | Soil | OVER | 0.602 | OVER | 0.145 | 0.223 | 0.359 |
| C | Hair gel | 2.976 | 0.398 | 2.898 | 0.142 | 0.165 | 0.207 |
| D | Hand sanitizer | 2.978 | 0.884 | 2.781 | 0.476 | 0.520 | 0.467 |
| E | Lotion | 2.938 | 0.425 | 2.805 | 0.334 | 0.409 | 0.478 |
| F | Saliva | 2.896 | 0.212 | 2.770 | 0.100 | 0.148 | 0.180 |
| G | Soap | 2.934 | 0.207 | 2.771 | 0.050 | 0.113 | 0.084 |
| H | N/A (Control) | 2.908 | 0.247 | 2.713 | 0.064 | 0.067 | 0.129 |

| | | | | | | |
|---|---|---|---|---|---|---|
| Precision CV %b | 1.66 | 41.04 | 1.80 | 47.04 | 47.04 | 47.04 |
| Calculated SDc | 0.04827 | 0.10137 | 0.04883 | 0.03011 | 0.03152 | 0.06068 |

a“OVER” results exceed the range of the DSX plate reader, values are greater than 3.0 AU and are considered high positives.
bCV % established from Precision tests.
cSD = CV %*Control/100

TABLE 2-12
Significance of potential interfering
substances on the HCV EIA test.

| Potential | | Significance | | | | | |
|---|---|---|---|---|---|---|---|
| Interference | | HCVP1 | HCVP2 | HCVP3 | N1 | N2 | N3 |
| A | Ethanol | N/A | 0 | 1 | 1 | 0 | 0 |
| B | Soil | N/A | 1 | N/A | 0 | 1 | 1 |
| C | Hair gel | 0 | 0 | 1 | 0 | 1 | 0 |
| D | Hand sanitizer | 0 | 1 | 0 | 1 | 1 | 1 |
| E | Lotion | 0 | 0 | 0 | 1 | 1 | 1 |
| F | Saliva | 0 | 0 | 0 | 0 | 0 | 0 |
| G | Soap | 0 | 0 | 0 | 0 | 0 | 0 |
| H | N/A (Control) | 0 | 0 | 0 | 0 | 0 | 0 |

Note:
“O” indicates differences between test samples and controls were less than ±3 SD, i.e. not significant. “1” indicates difference were larger than ±3 SD, i.e. significant.

Cross-reactivity of HCV assay to HIV, HSV-2 and Syphilis. In order to determine if the HCV assay is cross-reactive to any of the other three disease types; HIV, HSV-2, and Syphilis, the HCV result values were compared between samples that were only HCV positive and samples that were dual positive for HCV and another type of disease. A total of 801 samples, with reference results for all four assays, was used in this analysis.

HCV cross-reactivity to HSV-2 signals. Among all the HIV− and Syphilis-samples, there were 173 HSV-2−/HCV− samples, 61 HSV-2+/HCV− samples, 65 HSV-2−/HCV+ samples, and 64 HSV-2+/HCV+ samples. Therefore, it was only possible to determine if HSV-2 positive signals affect the background of the HCV assay. Analysis bins were set up according to the HCV OD value for a total range of 0 to 3.5, The frequency of samples in each analysis bin was plotted against bin values. Sample distribution was comparable between HSV-2+/HCV− and HSV-2−/HCV− groups, and between HSV-2+/HCV+ and HSV-2−/HCV− groups. Therefore, it is evident that HSV-2 positive signals do not affect HCV signals.

HCV's cross-reactivity to Syphilis signals. Among all the HIV− and HSV-2-samples, there were 173 Syph−/HCV− samples, 6 Syph+/HCV− samples, 65 Syph−/HCV+ samples, and 0 Syph+/HCV+ samples. Therefore, it was only possible to determine the influence of Syphilis disease state to HCV background signals. Analysis bins were set up according to the Syphilis Index for a total range of 0 to 3.5. The frequency of samples in each index bin was plotted against their Index value. Distributions were comparable between Syph−/HCV− and Syph−/HCV+ samples. This indicates that the Syphilis signal does not crosstalk to HCV antibody/antigens.

HCV's cross-reactivity to HIV signals. Because HSV-2 and Syphilis positive signals have not been found to interfere with the HCV EIA signals, all samples, including HSV-2 and/or Syphilis positive samples, were used to assess the interaction between HCV and HIV. There were 249 HIV−/HCV− samples, 264 HIV+/HCV− samples, 132 HIV−/HCV+ samples, and 65 HIV+/HCV+ samples. Analysis bins were set up according to the HCV OD value for a total range from 0 to 3.2. The frequency of samples in each bin was plotted against the bin value. 23.1% of HIV+/HCV+ samples had an OD value lower than 2, compared to 6.1% for the HIV−/HCV+ samples. HIV+/HCV+ samples have generally higher OD values than HIV−/HCV− samples. These results suggest that positive HIV disease status can affect HCV antibody titer. As a result, samples tested positive for both HIV and HCV need to be interpreted with caution.

In conclusion, the ORTHOR HCV Version 3.0 ELISA Test System has successfully been validated on the DSX with patient self-collected Dried Blood Spot cards and is ready for clinical implementation. This assay achieved 95.58% sensitivity and 98.92% specificity based on 412 patient samples collected on DBS cards (GE Whatman™ 903 protein saver cards). For all the positive samples tested the inter-day precision exhibited a CV less than 33% and the intra-day precision exhibited a CV less than 20%. DBS samples have been shown to be stable for a minimum of 8 weeks, post-generation, when stored at room temperature. Interference was observed from common substances such as 1% ethanol, soil samples, hand sanitizer and lotion. Additionally, HIV+ patients tend to have higher HCV background signals and lower positive HCV signals, which means that HIV+ samples need to have a second review on HCV results.

### HSV-2 Assay

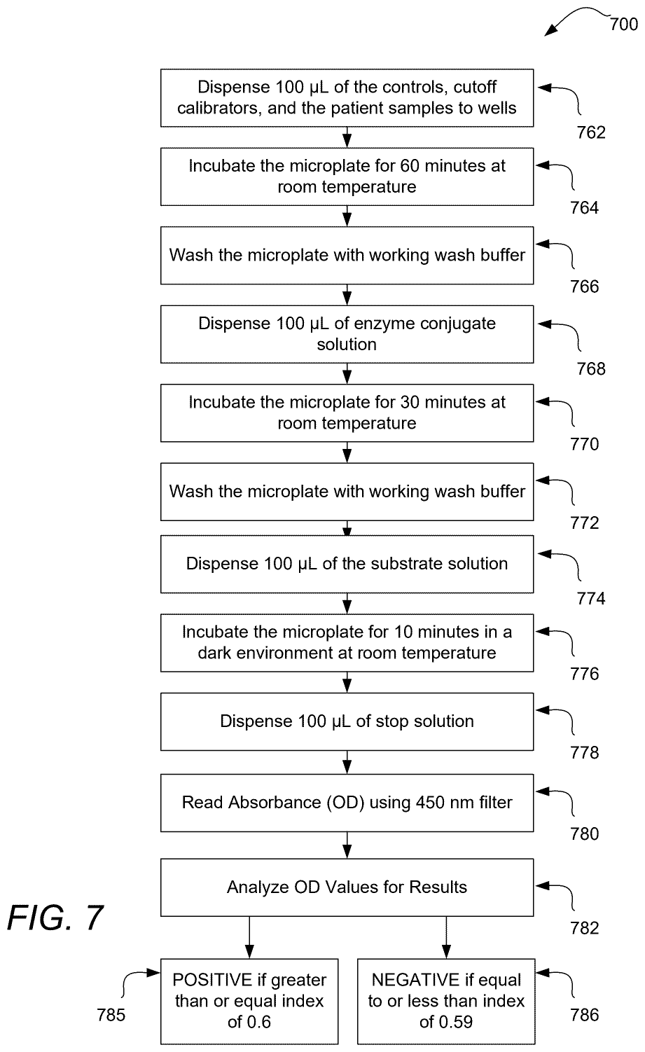

FIG. 7 is a method diagram 700 of specific DBX protocols for preparing an elution for an instrument prepared for an HSV-2 assay to detect the presence of HSV-2 from human dried blood spots collected remotely according to embodiments of the subject matter disclosed herein. Focus Diagnostics' HerpeSelect® 2 ELISA IgG test is tested for qualitatively detecting the presence or absence of human IgG class antibodies to HSV-2 in human sera or plasma. By changing the sample type from serum/plasma, which requires the collection of blood by a phlebotomist, to dried blood spot cards, this allows individuals to easily collect samples at home. This product is intended to provide an accessible, simple, at-home HSV-2 screening test to individuals who are at the risk of being exposed to STI.

Herpes simplex virus (HSV) is a common human pathogen found worldwide which produces a wide variety of diseases. The herpes simplex virus has been characterized into 2 distinct serotypes: HSV-1 and HSV-2. They both transmitted through direct contact with infected secretions from either a symptomatic or an asymptomatic host.

HSV-2 infections cause approximately 85% of symptomatic primary genital HSV cases, with HSV-1 infections causing the remainder. Since HSV-1 is unlikely to produce recurrent genital infections, 99% of recurrent genital herpes is due to HSV-2 infection. The classic clinical presentation of HSV2 infection is herpes genitalis, an infection characterized by bilaterally distributed lesions in the genital area accompanied by fever, inguinal lymphadenopathy, and dysuria. Genital ulcers have been associated with an increased risk for spreading HSV-2 infection.

HSV-2 infections acquired primarily through sexual contact. It can also be transmitted from an infected mother to her fetus/child and then develop into a neonate infection. HSV-2 infection is a life event that cannot be completely removed from the body and testing is the only way an individual can know of their HSV-2 status for sure; therefore, getting tested can provide important information to help keep themselves and others safe (HSV2 package insert).

Diagnosing HSV-2 can often be done simply through identifying the symptoms. However, the majority of people with herpes will have no symptoms, or the symptoms will be very mild. Many don't ever get diagnosed as the symptoms are mistaken for other causes. If obvious symptoms are present, a sample can be taken directly from the sores to be tested by viral culture, antigen detection or PCR. For general screening with absent symptoms, however, serological tests are the common method, with antibody screening of the sera being the primary method. (CDC Genital Herpes Fact Sheet, Mayo Clinic-Genital herpes). The Focus Diagnostics' HerpeSelect® 2 ELISA IgG test is an antibody screening test and can be used in aiding for the diagnosis for HSV-2.

The method 700 depicted in FIG. 7 has been validated as discussed below. Prior to discussing validation, the steps of the method are presented. In this method, once an elution of DBS chips is created, step 762 commences a technician dispensing 100 μL of the controls and the patient samples into a dilute in a microwell on a microplate. Next, at step 764, the technician may incubate the microplate for 60 minutes at room temperature followed by washing the microplate with a working wash buffer at step 766. This three-step sub-process may be repeated as step 768 continues with dispensing 100 μL of an enzyme conjugate into each of the microwell on the microplate. Next, at step 770, the technician may incubate the microplate for 30 minutes at room temperature followed by washing the microplate with a working wash buffer at step 772. This three-step sub-process may be repeated again as step 774 continues with dispensing 100 μL of a working substrate solution into each microplate well. Next, at step 776, the technician may incubate the microplate for 10 minutes in a dark environment at room temperature, however, without following up with any washing.

After the multiple sets of steps, the microplate may be read after step 778 whereby 100 mL of stop solution is dispensed into each microwell of the microplate. A technician at step 780 may read Absorbance (OD) using 450 nm filter to analyze Optical Density (OD) Values for results using cutoff calibrator at step 782. If the read OD value is greater than or equal to an index of 0.6, then the determination is a positive result at step 785 (e.g., determine the presence of HSV-2). However, If the read OD value less than an index of 0.59, then the determination is a negative result at step 786 (e.g., determine lack of presence of HSV-2).

The method 700 of FIG. 7 has been validated for precision, also referred to as reproducibility, which is a statistical measure of the variation between repeated test results on the same sample. Three types of variations were analyzed; Intra-run variation, i.e., repeats within the same run; intra-day variation, i.e., between runs, and inter-day variation, i.e., from day to day. The statistics conventionally used to express the precision profile of an assay are the mean (X), standard deviation (SD), and the coefficient of variation (CV); which is calculated by dividing the SD by the mean and multiplying by 100 to express it as a percentage.

$$CV ( \% ) = ( SD / X ) \times 100.$$

Eight unique patient samples were selected for validation of the precision of the assay. For each of the 8 patient samples 10 DBS cards generated in-house were utilized. In order to construct a well-represented precision profile for the HSV-2 EIA, all 8 precision samples were run in duplicate for a total of 20 days. All the data of each sample over 20 days was used in the calculation of the intra-run variation and inter-day variation according to Clinical and Laboratory Standards Institute guideline: CLSI EPV5-A2. Samples were run twice on an additional day, with each sample in triplicate on each run. For each sample, all 6 data points were used in the calculation of the intra-day variation. In order to demonstrate the reproducibility of the HSV-2 assay, runs were executed by alternating testing personnel at different times of the day, i.e. morning and afternoon runs, for the entire duration of the precision testing period.

The method 700 of FIG. 7 has been validated for adjustments to cutoff and accuracy. The preliminary result of the serological tests is the optical density (OD). However, in order to determine the diagnostic sensitivity and specificity of these assays, the test results must first be qualitatively identified as positive (detected) or negative (undetected). For the HSV-2 assay this OD value is first divided by the mean OD value of the Cutoff Calibrator included in every run. This ratio is referred to as the Index. According to the kit instructions, samples that have an Index value larger than 0.6 are considered as positive, while samples that have an Index value less than 0.59 are considered as negative. Index values between 0.59 and 0.6 are considered equivocal.

While this formula was made for serum/plasma samples, the DBS samples have diluted concentrations of antibodies/antigens compared to standard serum/plasma samples; therefore, the cutoff for DBS samples needed to be adjusted. A total of 794 self-collected DBS cards, including 395 HSV-2 positive samples and 399 HSV-2 negative samples, were tested. New cutoff values were selected to achieve the best sensitivity and specificity combination.

The method 700 of FIG. 7 has been validated for linearity. Due to the fact that the antibody concentration in the DBS elution is lower than serum/plasma samples, the cutoffs must be lowered. The validity of this strategy relies on the assay producing linear results when the antibody concentrations are lower than what is in the low titer control; which was in turn used to calculate the Index value of the samples. To ensure that the HSV-2 assay remains linear at lower concentrations, a linearity study was performed. The low titer control, with the original concentration considered as 1×, was diluted with elution buffer to 0.8×, 0.6×, 0.4×, 0.2× and 0.05× concentrations. Each dilution was repeated three times independently. All the diluted low filter controls, together with undiluted low titer control, were measured. The average of the OD values and the Index value were plotted to determine the linearity of this assay.

The method 700 of FIG. 7 has been validated for stability. A total of five samples were utilized to identify the stability of DBSs for the HSV-2 EIA test; five cards were generated from patient blood with known HSV-2 positive status. All of the cards were stored at room temperature. Tests were run in Week 0 (within three days of the DBS card generation), Week 1, Week 2, Week 4, Week 6, Week 8, and Week 12. All of the OD values were plotted against the time of testing and regression analysis was done using Microsoft Excel. A p-value larger than 0.05 suggests that no significant degradation occurred during the testing period.

The method 700 of FIG. 7 has been validated for assessment of carryover. To determine the possibility of using one puncher to punch multiple separate sample cards, the influence (carryover) of high-positive patient samples to subsequently punched negative patient samples was tested. One clean puncher was used to punch a known high-positive patient sample 20 times, then this same “dirty” puncher was used to punch a known negative patient sample five times. Each of these five negative punches were collected into five separated test tubes for elution and testing. These five punches were labeled as test punches according to the order of generation as “Test Punch-1” to “Test Punch-5”. A punch of this known positive sample was included as the positive control, while a punch of this known negative sample made by a clean puncher was used as the negative control. Carryover was assessed by determining the differences between the test punches and the clean negative control. In order to statistically calculate the differences, five known positive samples were paired with five known negative samples, and five sets of test punches were made and tested. Student t-test (one tail, type one) was used to analyze the differences.

The method 700 of FIG. 7 has been validated for interferences. Seven potential interfering substances were identified as commonly encountered substances during the collection of dried blood spot sample material. These 7 substances are outlined in Table 3-1. Each substance was dissolved into elution buffer (e.g., in one embodiment, PBS+ 0.05% TWEEN-20) to a 1% dilution. These solutions were utilized within 18 hours of generation and stored at 4° C. when not in use. Three unique, known-positive samples and three known-negative samples were chosen and eluted utilizing each of the seven test solutions. Blank elution buffer was used as a negative control.

TABLE 3-1
Overview of potential interfering substance
for the interference test.

| ID | Substance | Description |
|---|---|---|
| A | Ethanol | 70% Ethanol |
| B | Soil | Common soil sample (Vancouver, WA) |
| C | Hair gel | Dep Sport Endurance Styling Gel |
| D | Hand sanitizer | Purell ® Advanced Hand Sanitizer |
| E | Lotion | Jergens ®Extra Dry Skin Moisturizer |
| F | Saliva | Human saliva |
| G | Soap | Softsoap ® Lavender & Chamomile Hand Soap |
| H | N/A (Control) | No treatment - Elution buffer |

The method 700 of FIG. 7 has been validated for cross-reactivity of HSV-2 assay to HIV, HCV, and Syphilis. In addition to testing potential interferences coming from commonly encountered substances, the potential for the HSV-2 assay to cross-react with any of the other 3 disease types tested, such as HIV, HCV, and Syphilis, was also determined. A total of 801 samples, which were tested on all 4 assays, were used in this analysis. When cross-reactivity of the HSV-2 EIA to HIV EIA signals was analyzed, all HIV−/HSV-2-samples were separated into 4 groups: HIV−/HSV-2−, HIV+/HSV-2−, HIV−/HSV-2+, and HIV+/HSV-2+. In each group, samples were ranked from the lowest index value to the highest index value. Analysis bins were then generated based on the HSV-2 index value for a total range from 0 to 10. The frequency of samples that fell into each index bin was calculated. The frequencies were plotted again Index bin values to allow easy visualization of sample distributions in each of the groupings. In the scenario where HIV+ disease type samples were to affect the HSV-2 test signals, the distribution of HIV+/HSV-2− and HIV−/HSV-2− would be clearly different. The same methodology was used to analyze the potential cross-reactivity of the HSV-2 assay to other disease types.

Results and discussion for Precision. All positive samples yielded an Inter-day CV %, an Intra-day CV % and an Intra-run CV % within the acceptable range (<33%). Refer to Table 3.2. Negative samples tended to have a larger CV %. However, negative samples did yield lower absolute-Index values; in general, lower values are associated with a large variation. Additionally, all of the EIA results were reported qualitatively, positive call vs. negative call. Of all the negative samples tested none of the results were above the cutoff, which would have hypothetically yielded false low positives. Taking these 2 factors into consideration it follows that the larger CV percentages remain acceptable for the HSV-2 assay.

TABLE 3-2
Summary of precision tests for HSV-2 EIA.

| Intra-Day Variation | | | | Inter-Day Variation | | | | Intra-Run Variation | | | |
|---|---|---|---|---|---|---|---|---|---|---|---|
| (6 repeats) Index | | | | (Index) | | | | (Index) | | | |
| Sample | Mean | SD | CV % | Sample | Mean | SD | CV % | Sample | Mean | SD | CV % |
| POS1103 | 2.006 | 0.091 | 4.50% | POS1103 | 2.71 | 0.556 | 20.52% | POS1103 | 2.71 | 0.232 | 8.58% |
| POS1107 | 5.037 | 0.218 | 4.30% | POS1107 | 6.051 | 0.997 | 16.48% | POS1107 | 6.051 | 0.346 | 5.72% |
| POS1113 | 2.665 | 0.248 | 9.30% | POS1113 | 3.535 | 0.781 | 22.10% | POS1113 | 3.535 | 0.205 | 5.80% |
| STD1280 | 2.763 | 0.235 | 8.50% | STD1280 | 3.803 | 0.749 | 19.69% | STD1280 | 3.803 | 0.247 | 6.50% |
| STD1284 | 4.976 | 0.346 | 6.90% | STD1284 | 6.119 | 0.965 | 15.76% | STD1284 | 6.119 | 0.599 | 9.79% |
| POS1108 | 0.502 | 0.07 | 13.90% | POS1108 | 0.668 | 0.128 | 19.17% | POS1108 | 0.668 | 0.072 | 10.81% |
| STD1247 | 0.162 | 0.04 | 24.80% | STD1247 | 0.262 | 0.115 | 43.89% | STD1247 | 0.262 | 0.08 | 30.55% |
| STD1277 | 0.156 | 0.021 | 13.20% | STD1277 | 0.244 | 0.077 | 31.58% | STD1277 | 0.244 | 0.058 | 23.91% |

Results and discussion of adjustment of the DBS Cutoff and Accuracy Analysis. A total of 794 patient-collected DBS samples were tested; 395 of these were positive according to the reference results; i.e., Quest Diagnostics or PAML. The package insert for the HSV-2 EIA determined the result by applying the Index (OD/Average of the Cutoff Calibrator) and comparing it to 2 constant cutoff values, the upper-cutoff and the lower-cutoff. For serum/plasma samples the upper-cutoff was set to 1.1 and the lower-cutoff was set to 0.9.
If the Index upper-cutoff (1.1), the test result is considered positive for HSV-2.
If the Index lower-cutoff (0.9), the test result is considered negative for HSV-2.
If lower-cutoff (0.9)<the Index<upper-cutoff (1.1), the test result is equivocal.

For the purposes of DBS samples, it was determined that only a single cutoff value would be used to determine “Positive” or “Negative” status, with no “Equivocal” range. The ideal cutoff value should achieve the best sensitivity and specificity combination for the test. To have a comprehensive evaluation of the cutoff value different scenarios were considered. For example, first, sample stability was taken into consideration. From the stability test it was determined that the DBS samples are stable for eight weeks at room temperature (refer to 3.4.4 for details); therefore, cutoffs were calculated separately for samples tested within eight weeks vs. all samples. Second, since cross-reactivity with HIV was noted in the HCV and Syphilis assays (see 2.4.6 and 4.4.7), it was worthwhile to look into the impact of HIV+ samples on the HSV-2 assay. Therefore, HIV− samples were also separated from the total samples. Overall, there were four sample groups in which the sensitivity and specificity of potential cutoffs were calculated: 1) all samples tested (n=794; 2) all samples tested within 8 weeks of generation (n=371), 3) all HIV− samples tested (n=448), and 4) HIV− samples tested within eight weeks of generation (n=138). For each scenario, the sensitivity and specificity for the DBS samples were calculated based on the application of various new cutoff values (Table 3-3), and ROC curves were drawn based on different sensitivities and specificities (shown below).

From the ROC curve, it was clear that samples from groups 2 and 4 yielded the best sensitivities and specificities, compared to samples from groups 1 and 3, these differences emphasize the importance of testing samples within eight weeks after generation. When the cutoff was set at 0.600, all samples tested within eight weeks of generation yielded a sensitivity of 95.7% and a specificity of 97.5% (Table 3-4); HIV− samples tested within 8 weeks of generation yielded a sensitivity of 97.9% and a specificity of 97.8% (Table 3-5).

TABLE 3-3
The sensitivity and specificity of the HSV-2 EIA based on different cutoff values.

| | | Total | Cutoff | | | | | | | | | | | |
|---|---|---|---|---|---|---|---|---|---|---|---|---|---|---|
| | | # | Value | 0.100 | 0.200 | 0.300 | 0.400 | 0.500 | 0.600 | 0.700 | 0.710 | 0.720 | 0.730 | 0.740 |
| [symbol] | [symbol] | 371 | Sensitivity | 1.000 | 1.000 | 1.000 | 0.990 | 0.981 | 0.957 | 0.938 | 0.933 | 0.933 | 0.933 | 0.928 |
| | | | Specificity | 0.099 | 0.574 | 0.864 | 0.963 | 0.963 | 0.975 | 0.981 | 0.981 | 0.981 | 0.981 | 0.981 |
| | | | 1-Specificity | 0.901 | 0.426 | 0.136 | 0.037 | 0.037 | 0.025 | 0.019 | 0.019 | 0.019 | 0.019 | 0.019 |
| | | 794 | Sensitivity | 1.000 | 0.995 | 0.992 | 0.982 | 0.977 | 0.957 | 0.939 | 0.936 | 0.934 | 0.934 | 0.931 |
| | | | Specificity | 0.097 | 0.416 | 0.626 | 0.746 | 0.820 | 0.885 | 0.925 | 0.928 | 0.930 | 0.935 | 0.945 |
| | | | 1-Specificity | 0.903 | 0.584 | 0.374 | 0.254 | 0.180 | 0.115 | 0.075 | 0.072 | 0.070 | 0.065 | 0.055 |
| [symbol] | [symbol] | 138 | Sensitivity | 1.000 | 1.000 | 1.000 | 1.000 | 1.000 | 0.979 | 0.936 | 0.936 | 0.936 | 0.936 | 0.915 |
| | | | Specificity | 0.099 | 0.637 | 0.912 | 0.967 | 0.967 | 0.978 | 0.989 | 0.989 | 0.989 | 0.989 | 0.989 |
| | | | 1-Specificity | 0.901 | 0.363 | 0.088 | 0.033 | 0.033 | 0.022 | 0.011 | 0.011 | 0.011 | 0.011 | 0.011 |
| | [symbol] | 448 | Sensitivity | 1.000 | 1.000 | 1.000 | 1.000 | 1.000 | 0.980 | 0.960 | 0.960 | 0.954 | 0.954 | 0.947 |
| | | | Specificity | 0.081 | 0.364 | 0.549 | 0.667 | 0.768 | 0.852 | 0.906 | 0.909 | 0.912 | 0.919 | 0.933 |
| | | | 1-Specificity | 0.919 | 0.636 | 0.451 | 0.333 | 0.232 | 0.148 | 0.094 | 0.091 | 0.088 | 0.081 | 0.067 |

[symbol]

indicates data missing or illegible when filed

TABLE 3-4
Comparison of in-house and reference lab HSV-2 test results
for all samples tested within 8 weeks after generation.

| | MTL | | |
|---|---|---|---|
| REFERENCE | Positive | Negative | Total |
| Positive | 200 | 9 | 209 |
| Negative | 4 | 158 | 162 |
| Total | 204 | 167 | 371 |

TABLE 3-5
Comparison of in-house and reference lab HSV-2 test results
for HIV− samples tested within 8 weeks after generation.

| | MTL | | |
|---|---|---|---|
| REFERENCE | Positive | Negative | Total |
| Positive | 46 | 1 | 47 |
| Negative | 2 | 89 | 91 |
| Total | 48 | 90 | 138 |

Results and discussion for linearity. The HSV-2 assay showed good linear signals in the range from 0.05× to 1× concentration of the Cutoff Calibrator, resulting an R-squared value 0.9903 for the linear regression (Table 3.5 and graph shown below). Because the assay still provides linear signals cross the cutoff zone that was re-defined for DBS samples, we conclude that it is valid to use lower index value to define positive/negative samples.

TABLE 3-6
HSV-2 Cutoff Calibrator Linearity Data.

| Dilution Ratio | 0.05 | 0.20 | 0.40 | 0.60 | 0.80 | 1.00 |
|---|---|---|---|---|---|---|
| Experiment 1 | 0.014 | 0.014 | 0.014 | 0.014 | 0.014 | 0.014 |
| Experiment 2 | 0.021 | 0.021 | 0.021 | 0.021 | 0.021 | 0.021 |
| Experiment 3 | 0.019 | 0.019 | 0.019 | 0.019 | 0.019 | 0.019 |
| OD Average | 0.018 | 0.073 | 0.157 | 0.196 | 0.297 | 0.344 |
| 00S0 | 0.004 | 0.005 | 0.011 | 0.014 | 0.013 | 0.012 |

Results and discussion for Stability. Five DBS samples were tracked over a period of 8 weeks. See Table 3.7. Regression analysis showed that none of the samples exhibited significantly decreased test signals within the allotted time frame. Therefore, it was concluded that for HSV-2 EIA testing, DBS samples are stable for at least eight weeks post-generation.

TABLE 3-7
Index values for all DBS samples tested on
the HSV-2 assay for stability purposes.

| | Samples | | | | | | | | | |
|---|---|---|---|---|---|---|---|---|---|---|
| | PSHS | POS | PSHS | POS | PSHS | POS | PSHS | POS | PSHS | POS |
| Week | 1044 | 1044 | 1047 | 1047 | 1053 | 1053 | 1055 | 1055 | 1057 | 1057 |
| 0 | 2.041 | 2.385 | 2.813 | 3.063 | 6.051 | 7.245 | 4.214 | 4.070 | 3.464 | 4.376 |
| 1 | 1.471 | 1.835 | 2.559 | 2.599 | 5.994 | 6.807 | 3.290 | 2.792 | 3.035 | 3.000 |
| 2 | 1.410 | 1.512 | 2.717 | 2.727 | 6.302 | 6.536 | 3.111 | 3.387 | 4.058 | 3.945 |
| 4 | 1.755 | 2.312 | 3.194 | 3.249 | 5.077 | 5.776 | 2.601 | 3.135 | 3.513 | 3.465 |
| 6 | 1.120 | 2.026 | 2.821 | 2.724 | 6.083 | 6.858 | 2.745 | 3.112 | 3.021 | 4.097 |
| 8 | 1.007 | 1.707 | 2.414 | 2.725 | 7.228 | 7.433 | 4.408 | 4.149 | 4.669 | 4.215 |
| p-value | 0.060 | 0.639 | 0.709 | 0.765 | 0.384 | 0.785 | 0.954 | 0.703 | 0.358 | 0.597 |

Results and discussion for Carryover. To determine the possibility of using one puncher to punch multiple separate sample cards, the influence (carryover) of high-positive patient samples to subsequently punched negative patient samples was tested. This experiment revealed that after generating 20 high-positive punches, i.e., punching 5 unique patient samples 4 times each, no carryover was identified. The relative signal changes of the test punches compared to the negative cards were small (Table 3-9). None of the negative samples punched after the high positives resulted in a significant increase in comparison to the original negative sample signal, refer to Table 3-8.

TABLE 3-8
Average OD values for all DBS samples tested in the carryover experiment.

| Positive | Positive | Negative | Negative | Test | Test | Test | Test | Test |
|---|---|---|---|---|---|---|---|---|
| Sample ID | Card OD | Sample ID | Card OD | punch-1 | punch-2 | punch-3 | punch-4 | punch-5 |
| STD1069 | OVER | STD1247 | 0.093 | 0.106 | 0.112 | 0.094 | 0.105 | 0.104 |
| STD1140 | OVER | STD1250 | 0.107 | 0.116 | 0.110 | 0.129 | 0.123 | 0.100 |
| STD1168 | OVER | STD1253 | 0.079 | 0.091 | 0.108 | 0.075 | 0.097 | 0.069 |
| STD1187 | OVER | STD1259 | 0.105 | 0.119 | 0.100 | 0.107 | 0.099 | 0.084 |
| STD1263 | OVER | STD1263 | 0.088 | 0.060 | 0.090 | 0.091 | 0.081 | 0.079 |
| | | | t-test | 0.326 | 0.098 | 0.172 | 0.136 | 0.117 |

TABLE 3-9
DBS test samples normalized to known-negative values (Sample OD/Known-Negative OD).

| Positive | Positive | Negative | Negative | Test | Test | Test | Test | Test |
|---|---|---|---|---|---|---|---|---|
| Sample ID | Card OD | Sample ID | Card OD | punch-1 | punch-2 | punch-3 | punch-4 | punch-5 |
| STD1069 | OVER | STD1247 | 1.000 | 1.134 | 1.199 | 1.005 | 1.129 | 1.113 |
| STD1140 | OVER | STD1250 | 1.000 | 1.089 | 1.033 | 1.211 | 1.150 | 0.939 |
| STD1168 | OVER | STD1253 | 1.000 | 1.146 | 1.361 | 0.949 | 1.222 | 0.873 |
| STD1187 | OVER | STD1259 | 1.000 | 1.134 | 0.947 | 1.019 | 0.947 | 0.804 |
| STD1283 | OVER | STD1261 | 1.000 | 0.680 | 1.029 | 1.040 | 0.926 | 0.903 |
| | | | Average | 1.037 | 1.114 | 1.045 | 1.075 | 0.926 |

Results and discussion for Interferences. The sensitivity of the HSV-2 EIA test to interferences was tested. The influence of 7 substances that are commonly encountered during DBS sample collection were focused on. Based on the established CV % from the precision study, an SD value was calculated for each sample at its signal level, refer to Table 3.10. The differences between samples containing and those without potential interfering substances were within ±3 SD range, refer to Table 3.11. Therefore, it has been concluded that the HSV-2 EIA test is not significantly affected by substances such as 1% of ethanol, soil, hair gel, hand sanitizer, lotion, saliva, and soap products.

TABLE 3-10
OD values for HSV-2 positive and negative samples tested utilizing
elution buffers with various potential interfering substances.

| Potential | | Ratio (S/CO) | | | | | |
|---|---|---|---|---|---|---|---|
| Interference | | HSV-2P1 | HSV-2P2 | HSV-2P3 | N1 | N2 | N3 |
| A | Ethanol | 5.626 | 4.606 | 5.724 | 0.224 | 0.193 | 0.207 |
| B | Soil | 5.652 | 4.898 | 6.125 | 0.167 | 0.192 | 0.237 |
| C | Hair gel | 5.671 | 4.895 | 5.878 | 0.138 | 0.146 | 0.262 |
| D | Hand sanitizer | 5.511 | 4.699 | 5.724 | 0.148 | 0.18 | 0.365 |
| E | Lotion | 4.861 | 3.600 | 5.755 | 0.065 | 0.127 | 0.098 |
| F | Saliva | 5.755 | 4.547 | 5.801 | 0.206 | 0.164 | 0.296 |
| G | Soap | 6.008 | 4.611 | 5.557 | 0.093 | 0.172 | 0.233 |
| H | N/A (Control) | 5.513 | 4.178 | 5.157 | 0.13 | 0.175 | 0.279 |

| | | | | | | |
|---|---|---|---|---|---|---|
| Precision CV %ₐ | 11.56 | 10.93 | 16.56 | 27.22 | 27.22 | 40.39 |
| Calculated SD_{b} | 0.637 | 0.457 | 0.854 | 0.035 | 0.048 | 0.113 |

ₐCV % established from Precision tests.
_{b}SD = CV %^{a} Control/100

TABLE 3-11
Significance of potential interfering substances on the HSV-2 test.

| Potential | | SIGNIFICANCE | | | | | |
|---|---|---|---|---|---|---|---|
| Interference | | HSV-2P1 | HSV-2P2 | HSV-2P3 | N1 | N2 | N3 |
| A | Ethanol | 0 | 0 | 0 | 0 | 0 | 0 |
| B | Soil | 0 | 0 | 0 | 0 | 0 | 0 |
| C | Hair gel | 0 | 0 | 0 | 0 | 0 | 0 |
| D | Hand sanitizer | 0 | 0 | 0 | 0 | 0 | 0 |
| E | Lotion | 0 | 0 | 0 | 0 | 0 | 0 |
| F | Saliva | 0 | 0 | 0 | 0 | 0 | 0 |
| G | Soap | 0 | 0 | 0 | 0 | 0 | 0 |
| H | N/A (Control) | 0 | 0 | 0 | 0 | 0 | 0 |

Note:
“O” indicates differences between test samples and controls were less than ±3S0, i e. not significant. “1” indicates difference were larger than ±3S0, i.e. significant.

Results and discussion for Cross-reactivity of HSV-2 assay to HIV, HCV and Syphilis. In order to determine if the HSV-2 assay is cross-reactive to the other 3 disease types, such as HCV, HIV and Syphilis, HSV-2 result value were compared between samples that were only HSV-2 positive and samples that were dual positive for HSV-2 and another type of disease. A total of 267 samples, with reference results for all 4 assays, was used in this analysis.

HSV-2's cross-reactivity to HIV signals. Among all the Syphilis− and HCV− samples, there were 218 HIV−/HSV-2− samples, 74 HIV+/HSV-2− samples, 77 HIV−/HSV-2+ samples, and 107 HIV+/HSV-2+ samples. Analysis bins were set up according to the HSV-2 Index for a total range from 0 to 10.0. The frequency of samples in each index bin was plotted against the index value. Sample distribution was comparable between HIV+/HSV-2+ and HIV−/HSV-2+ groups. This indicates that the HSV-2 assay does not crosstalk to HIV antibody/antigens. However, HIV+/HSV-2− samples generally had lower HSV-2 Index than HIV−/HSV-2− samples. Therefore, positive HIV disease states should not affect the establishment of new HSV-2 cutoffs. This is shown in the graph below.

HSV-2's cross-reactivity to HCV signals. Among all the Syphilis− and HIV− samples, there were 218 HCV−/HSV-2− samples, 62 HCV+/HSV-2− samples, 77 HCV−/HSV-2+ samples, 64 HCV+/HSV-2+ samples. Analysis bins were set up according to the HSV-2 Index for a total range from 0 to 10.0. The frequency of samples in each index bin were plotted against the index value. Sample distribution was comparable between HCV+/HSV-2+ and HCV−/HSV-2+ groups. This indicates that the HSV-2 assay does not crosstalk to HCV antibody/antigens. However, HCV+/HSV-2− samples generally had lower HSV-2 Index values than HCV−/HSV-2− samples. Therefore, HCV disease states should not affect the establishment of new HSV-2 cutoffs. This is shown in the graph below.

HSV2's cross-reactivity to Syphilis signals. Among all the HIV− and HCV− samples, there were 219 Syph−/HSV-2− samples, 7 Syph+/HSV-2− samples, 78 Syph−/HSV2+ samples, and 4 Syph+/HSV-2+ samples. Due to the insufficient sample counts for Syph+/HSV-2− and Syph+/HSV-2+ groups, HSV-2 cross-reactivity to syphilis was not defined in this study.

In conclusion, the HerpeSelect® 2 ELISA IgG has successfully been validated on the DSX with self-collected Dried Blood Spot cards and is ready for clinical implementation. This assay achieved 95.7% sensitivity and 97.5% specificity based on 371 patient samples collected on DBS cards (GE Whatman™ 903 protein saver cards). The inter-day and intra-day precision exhibited a CV less than 33%. DBS samples have been shown to be stable for a minimum of eight weeks, post-generation, when stored at room temperature. Additionally, no interference was observed from common substances such as 1% ethanol, soil samples, hand sanitizer, lotion, saliva, and soap products. No cross-reactivity has been found between HSV-2 and HIV, and between HSV-2 and HCV.

### Syphilis Assay

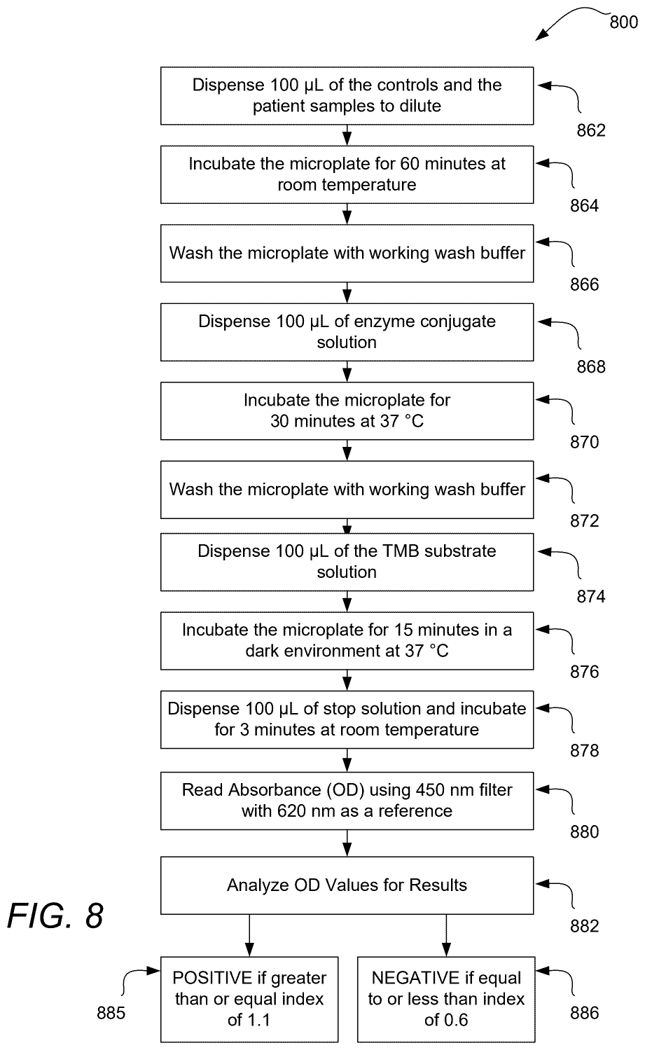

FIG. 8 is a method diagram 800 of specific DBX protocols for preparing an elution for an instrument prepared for a syphilis assay to detect the presence of syphilis from human dried blood spots collected remotely according to embodiments of the subject matter disclosed herein. CAPTIA™ Syphilis (T. pallidum)-G and Trep-Sure™ EIA are an enzyme immunoassay kit for the qualitative detection of antibodies to T. pallidum in human serum or plasma. It is intended for screening blood and/or plasma donors to identify a past or current infection of syphilis. By changing the sample type from serum/plasma to dried blood spot cards, this allows individuals to more easily collect samples at home, rather than requiring a phlebotomist to collect whole blood samples. This product is intended to provide an accessible, simple, at-home Syphilis screening test to individuals who are at the risk of being exposed to STI.

Syphilis is a disease, usually sexually transmitted, and caused by an infection with the spirochete Treponema pallidum (T. pallidum). There are 4 stages of Syphilis, each stage exhibiting different symptoms. The first sign of syphilis (primary syphilis) is a small sore, called a chancre. The chancre usually develops about 3 weeks after exposure, and then heals on its own within 6 weeks. Within a few weeks of the original chancre healing, the infected individual may experience new symptoms (secondary syphilis) such as a whole-body rash, and/or muscle aches, fever, sore throat and swollen lymph nodes. These signs and symptoms may disappear within a few weeks or repeatedly come and go for as long as a year. If the syphilis infection is not treated, the disease moves from the secondary stage to the latent stage, with no symptoms (Latent syphilis). About 15% to 30% of infected people, after years of latent stage, develop complications known as tertiary (late) syphilis. In the late stages, the disease may damage the brain, nerves, eyes, heart, blood vessels, liver, bones, and joints (Mayo Clinic Syphilis).

Syphilis is systemic from the onset of initial infection and the disease is characterized by periods of latency, often up to 20 years. These factors, coupled with the difficulty in isolating T. pallidum from cultures mean that serological techniques play a major role in the diagnosis of syphilis and treatment follow-up (Syphilis package insert).

In clinical diagnostic laboratories, the procedures most commonly used to screen for antibodies to T. pallidum are based upon their reaction with non-treponemal lipoidal antigens (the reagin tests). Reagin tests, such as the RPR or VDRL, can be used to test serial dilutions of the serum specimen. The endpoint values from sequentially obtained serum samples decline after successful treatment. After several months of successful treatment, the patient will usually become reagin non-reactive.

Clinical diagnostic serum specimens which are reactive in regain test are typically confirmed using treponemal tests such as the Microhaemagglutination-T. pallidum (MHA-TP), or the Fluorescent Treponemal Antibody-Absorption (FTA-ABS) test. The CAPTIA™ Syphilis (T. pallidum)-G kit tested validated is a treponemal test. In contrast to the non-treponemal tests, treponemal test reactivity will persist following treatment. In approximately 85% of the cases the reactivity lasts for the life of the infected person. The combination of a quantitative non-treponemal test (such as RPR or VDRL) and a treponemal test allows for distinction between active and past infections and assists in ruling out false positives (Syphilis package insert).

The method 800 depicted in FIG. 8 has been validated as discussed below. Prior to discussing validation, the steps of the method are presented. In this method, once an elution of DBS chips is created, step 862 commences wherein a technician dispenses 100 μL of the controls, cutoff calibrators, and the patient samples into a microwell on a microplate. Next, at step 864, the technician may incubate the microplate for 60 minutes at room temperature followed by washing the microplate with a working wash buffer at step 866. This three-step sub-process may be repeated as step 868 continues with dispensing 100 μL of an enzyme conjugate into each the microwell on the microplate. Next, at step 870, the technician may incubate the microplate for 60 minutes at 37° C. followed by washing the microplate with a working wash buffer at step 872. This three-step sub-process may be repeated again as step 874 continues with dispensing 100 μL of the substrate solution into each microplate well. Next, at step 876, the technician may incubate the microplate for 30 minutes in a dark environment at room temperature, however, without following up with any washing.

After the multiple sets of steps, the microplate may be read after step 878 whereby 100 mL of stop solution is dispensed into each microwell of the microplate followed by an incubation of the plate for three minutes at room temperature. A technician at step 880 may read Absorbance (OD) using 450 nm filter with 620 nm as a reference to analyze Optical Density (OD) Values for results at step 882. If the read OD value is greater than or equal to an index of 1.1, then the determination is a positive result at step 885 (e.g., determine the presence of syphilis). However, If the read OD value less than an index of 0.6, then the determination is a negative result at step 886 (e.g., determine lack of presence of syphilis).

In a related embodiment not shown in any FIG. a different reagent may also be used with similar methodology, Thus, the Trinity Biotech Trep-Sure™ EIA (Trinity #TS-96) enzyme immunoassay kit for the qualitative detection of antibodies to T. pallidum in human serum or plasma has been validated as discussed below. In this method, once an elution of DBS chips is created, a technician dispenses 100 μL of the controls, cutoff calibrators, and the patient samples into a microwell on a microplate. Next, the technician may incubate the microplate for 60 minutes at room temperature followed by washing the microplate with a working wash buffer. This three-step sub-process may be repeated with dispensing 100 μL of an enzyme conjugate into each the microwell on the microplate. Next, the technician may incubate the microplate for 30 minutes at 37° C. followed by washing the microplate with a working wash buffer. This three-step sub-process may be repeated again with dispensing 100 μL of TMB substrate solution into each microplate well. Next, the technician may incubate the microplate for 15 minutes in a dark environment at 37° C., however, without following up with any washing.

After the multiple sets of steps, the microplate may be read whereby 100 ml of stop solution is dispensed into each microwell of the microplate. A technician 880 may read Absorbance (OD) using 450 nm filter with 620 nm as a reference to analyze Optical Density (OD) Values indicate results wherein if the read OD value is greater than or equal to an index of 1.1, then the determination is a positive result (e.g., determine the presence of syphilis). However, If the read OD value less than an index of 0.6, then the determination is a negative result at step 886 (e.g., determine lack of presence of syphilis).

The method 800 of FIG. 8 has been validated for precision, also referred to as reproducibility, which is a statistical measure of the variation between repeated test results on the same sample. Three types of variations were analyzed; Intra-run variation, i.e., repeats within the same run; intra-day variation, i.e., between runs, and inter-day variation, i.e., from day to day. The statistics conventionally used to express the precision profile of an assay are the mean (X), standard deviation (SD), and the coefficient of variation (CV); which is calculated by dividing the SD by the mean and multiplying by 100 to express it as a percentage.

$$CV ( \% ) = ( SD / X ) \times 100$$

Thirteen total samples were selected for validation of the precision of the Syphilis assay; this includes eight unique patient samples and five synthetic samples. For each of these samples, ten DBS cards generated in-house were utilized. In order to construct a well-represented precision profile for the Syphilis EIA, all precision samples were run in duplicate for a total of 20 days. All the data of each sample over 20 days was used in the calculation of the intra-run variation and inter-day variation according to Clinical and Laboratory Standards Institute guideline. Samples were run twice on an additional day, with each sample in triplicate on each run. For each sample all 6 data points were used in the calculation of the intra-day variation. In order to demonstrate the reproducibility of the Syphilis assay, runs were executed by alternating testing personnel at different times of the day, i.e., morning and afternoon runs, for the entire duration of the precision testing period.

The method 800 of FIG. 8 has been validated for adjusting the cutoff and accuracy. The preliminary result of the serological test is the optical density (OD). However, in order to determine the diagnostic sensitivity and specificity of these assays, the test results must first be qualitatively identified as positive (detected) or negative (undetected). For the Syphilis assay, the OD values of samples or controls are first divided by the mean OD value of the Low Titer Reactive controls included in every run. This ratio is called a sample Index. Samples that have an Index value greater than 1.1 (an upper cutoff) are considered as positive, while samples that have an Index value less than 0.9 (a lower-cutoff) are considered as negative. Index values between 0.9 and 1.1 are considered equivocal and require a retest to be done prior to distributing a final call.

While this formula was generated for serum/plasma samples, the DBS samples have diluted concentrations of antibodies/antigens in comparison to standard serum/plasma samples. In order to compensate for the differing sample type, the upper and lower cutoffs for the DBS samples must be adjusted. A total of 892 self-collected DBS cards, including 135 Syphilis positive samples and 757 Syphilis negative samples, were tested. New cutoff values were selected to achieve the best sensitivity and specificity combination.

As previously mentioned, both non-treponemal tests and treponemal tests are commonly used to screen/determine Syphilis infections. Participant samples collected at ALTN were tested for Syphilis predominately with RPR testing through a reference lab; some concurrently had FTA-ABS testing done. SetPoint participant samples were tested by EIA and then RPR testing, with confirmatory testing done with the TPPA test. Our final reference result for each patient was based on evaluation of all available test results. For instance, if one patient's RPR result was negative, but the FTA-ABS or TPPA result was positive, the final result was considered as positive, for reference purposes. The logic behind this type of call is because FTA-ABS and/or TPPA tests are more similar to the CAPTIA™ Syphilis assay in comparison to RPR testing. All the tests detect the treponemal specific antibodies, except for RPR test.

The method 800 of FIG. 8 has been validated for Linearity. Due to the fact that the antibody concentration in the DBS elution is lower than serum/plasma samples, the upper-cutoff and lower-cutoff must be lowered. The validity of this strategy relies on the assay producing linear results when the antibody concentrations are lower than what is in the low titer control, which was used to calculate the Index value of the samples. To ensure that the Syphilis assay remains linear at lower concentrations, a linearity study was performed. Specifically, the low titer control, with the original concentration considered as 1×, was diluted with elution buffer (e.g., in one embodiment, 1×PBS+0.05% TWEEN-20) to 0.8×, 0.6×, 0.4×, 0.2× and 0.05× concentrations. Each dilution was repeated three times independently. All the diluted low filter controls, together with undiluted low titer control, were measured. The average of the OD values and the Index value were plotted to determine the linearity of this assay.

The method 800 of FIG. 8 has been validated for Stability. A total of 12 samples were utilized to identify the stability of DBS for the Syphilis EIA test; five cards were generated from synthetic blood, and seven cards were generated from patient blood. All of the cards were stored at room temperature. Tests were run in Week 0 (within three days of the DBS card generation), Week 1, Week 2, Week 4 (or Week 5 when the Week 4 data was affected), Week 6 and Week 8. All of the Index values were plotted against the time of testing and regression analysis was done using Microsoft Excel. A p-value larger than 0.05 suggests that no significant degradation occurred during the testing period.

The method 800 of FIG. 8 has been validated for Carryover. To determine the possibility of using one puncher to punch multiple separate sample cards, the influence (carryover) of high-positive patient samples to subsequently punched negative patient samples was tested. One clean puncher was used to punch a known high-positive patient sample 20 times. This same “dirty” puncher was then used to punch a known negative patient sample five times. Each of these five negative punches were collected into 5 separated test tubes for elution and testing. These five punches were labelled as test punch according to the order of generation as “Test Punch-1” to “Test Punch-5”. A punch of this known positive sample was included as the positive control, while a punch of this known negative sample made by a clean puncher was used as the negative control. Carryover was assessed by determining the differences between the test punches and the clean negative control. In order to statistically calculate the differences, five known positive samples were paired with five known negative samples, and five sets of test punches were made and tested. Student t-test (one tail, type one) was used to analyze the differences.

The method 800 of FIG. 8 has been validated for Interferences. Seven potential interfering substances were identified as commonly encountered substances during the collection of dried blood spot sample material. See Table 4.1. Each substance was dissolved into elution buffer (e.g., in one embodiment, PBS+0.05% TWEEN-20) to a 1% dilution. These solutions were utilized within 18 hours of generation and stored at 4° C. when not in use. Three unique, known-positive samples and three known-negative samples were chosen and eluted utilizing each of the seven test solutions. Blank elution buffer was used as a negative control.

TABLE 4-1
Overview of potential interfering substance
for the interference test.

| ID | Substance | Description |
|---|---|---|
| A | Ethanol | 70% Ethanol |
| B | Soil | Common soll sample (Vancouver, WA) |
| C | Hair gel | Dep Sport Endurance Styling Gel |
| D | Hand sanitizer | Purell ® Advanced Hand Sanitizer |
| E | Lotion | Jergens ® Extra Dry Skin Moisturizer |
| F | Saliva | Human saliva |
| G | Soap | Softsoap ® Lavender & Chamomile Hand Soap |
| H | N/A (Control) | No treatment - Elution buffer |

The method 800 of FIG. 8 has been validated for Cross-reactivity of Syphilis assay to HIV, HCV and HSV-2. In addition to testing potential interferences coming from commonly encountered substances, we also determined if the Syphilis assay cross-reacted with any of the other three disease types tested, such as HIV, HCV and HSV-2. A total of 267 samples that were tested on all four assays was used in this analysis. For instance, when cross-reactivity of the Syphilis EIA to HIV EIA signals was analyzed, all HCV−/HSV-2− samples were separated into four groups: HIV−/Syph−, HIV+/Syph−, HIV−/Syph+, and HIV+/Syph+. In each group, samples were ranked from the lowest Index value to the highest Index value. Analysis bins were then generated based on Syphilis Index value for a total range from 0.05 to 2.0. From 0.05 to 1.55, each bin differs by 0.05 increments. Index values great than 1.55 were distributed into 2 final bins. The first held all values ranging from 1.55-2.0, the second bin contained values >2.0. The frequency of samples that fell into each index bin was calculated. The frequencies were plotted again

Index bin values to allow easy visualization of sample distributions in each of the groupings. If HIV+ disease type samples were to affect the Syphilis test signals, the distribution of HIV+/Syph− and HIV−/Syph− would be clearly different. The same methodology was used to analyze the potential cross-reactivity of the Syphilis assay to other disease types.

The method 800 of FIG. 8 has been validated for comparisons between DSX method and semi-automatic method. From previous experience, it was realized that when the CAPTIA™ Syphilis assay was run on the DSX it generated a higher background than when it was run with the semi-automatic method. In order to determine whether the semi-automatic method can help solve the background issue, a direct comparison between DSX and semi-automatic methods was performed. Eleven positive samples, including three HIV single positive, three HSV-2 single positive, three HCV single positive, and two Syphilis single positive samples, as well as six negative samples were used in this study. Elution of each sample was divided into two tubes: one for the DSX and the other for the semi-automatic method. Paired student t-test was used to analyze the differences of Syphilis negative sample results generated by the two different methods.

Results and discussion for Precision. Because Syphilis is initially measured as an OD value and then calculated into an Index value (Sample OD/Mean Low Titer Control), the Inter-day CV %, the Intra-day CV % and Intra-run CV % were calculated twice, using each type of sample value (the OD and the Index value). The CV % of all precisions in OD measurements of all samples were within the acceptable range (<33%). See Table 4.2.

TABLE 4-2
Summary of all precision tests for the Syphilis EIA based on OD values.

| Intra-Day Variation (OD) | | | | Inter-Day Variation (OD) | | | | Intra-Run Variation (OD) | | | |
|---|---|---|---|---|---|---|---|---|---|---|---|
| Sample | Mean | SD | CV % | Sample | Mean | SD | CV % | Sample | Mean | SD | CV % |
| POS1011 | 0.68 | 0.048 | 7.00% | POSI0II | 0.801 | 0.193 | 24.11% | POSIOII | 0.801 | 0.052 | 6.44% |
| POS1012 | 1.072 | 0.082 | 7.70% | POS1012 | 1.062 | 0.248 | 23.40% | POS1012 | 1.062 | 0.082 | 7.74% |
| POS1024 | 0.508 | 0.038 | 7.50% | POS1024 | 0.557 | 0.154 | 27.66% | POS1024 | 0.557 | 0.042 | 7.50% |
| POS1052 | 1.266 | 0.074 | 5.90% | STD1052 | 1.389 | 0.224 | 16.10% | STD1052 | 1.389 | 0.059 | 4.25% |
| POS1109 | 0.673 | 0.06 | 8.80% | POS1109 | 0.766 | 0.172 | 22.40% | POS1109 | 0.766 | 0.048 | 6.26% |
| STD1262 | 0.614 | 0.044 | 7.10% | STD1262 | 0.756 | 0.174 | 23.05% | STD1262 | 0.756 | 0.046 | 6.03% |
| STD1253 | 0.109 | 0.016 | 14.50% | STD1253 | 0.117 | 0.031 | 26.51% | STD1253 | 0.117 | 0.01 | 8.85% |
| STD1259 | 0.176 | 0.015 | 8.30% | STD1259 | 0.192 | 0.043 | 22.66% | STD1259 | 0.192 | 0.018 | 9.16% |
| sSyph-02 | 0.922 | 0.052 | 5.60% | sSyph-02 | 1.047 | 0.192 | 18.30% | sSyph-02 | 1.047 | 0.045 | 4.34% |
| sSyph-06 | 0.727 | 0.062 | 8.50% | sSyph-06 | 0.841 | 0.148 | 17.59% | sSyph-06 | 0.841 | 0.038 | 4.46% |
| sSyph-10 | 0.994 | 0.078 | 7.80% | sSyph-10 | 1.098 | 0.197 | 17.98% | sSyph-10 | 1.098 | 0.041 | 3.75% |
| sSyph-16 | 0.772 | 0.071 | 9.20% | sSyph-16 | 1.01 | 0.218 | 21.60% | sSyph-16 | 1.01 | 0.142 | 14.07% |
| sSyph-21 | 0.593 | 0.041 | 6.90% | sSyph-21 | 0.766 | 0.188 | 24.60% | sSyph-21 | 0.766 | 0.143 | 18.67% |

While the CV % of the Intra-run and Intra day index value of all samples was smaller than 20%, the CV % of the Inter-day Index values of positive samples was around 30%-40%, slightly larger than what was expected. Furthermore, the CV % of the Inter-day Index values for the negative samples were even greater than that of the positive samples, as high as 46%. See Table 4.3.

TABLE 4-3
Summary of all precision tests for the Syphilis EIA based on Index values.

| Intra-Day Variation (Index) | | | | Inter--Day Variation (Index) | | | | Intra-Run Variation (Index) | | | |
|---|---|---|---|---|---|---|---|---|---|---|---|
| Sample | Mean | SD | CV % | Sample | Mean | SD | CV % | Sample | Mean | SD | CV % |
| POSIOII | 3.225 | 0.229 | 7.10% | POSIOII | 1.876 | 0.569 | 30.34% | POSIOII | 1.876 | 0.183 | 9.789% |
| POS1012 | 5.106 | 0.632 | 12.40% | POS1012 | 2.5 | 0.798 | 31.93% | POS1012 | 2.5 | 0.269 | 10.76% |
| POS1024 | 2.42 | 0.291 | 12.00% | POS1024 | 1.308 | 0.441 | 33.73% | POS1024 | 1.308 | 0.113 | 8.67% |
| POS1052 | 6.026 | 0.588 | 9.80% | POS1052 | 3.333 | 1.224 | 36.73% | STD1052 | 3.333 | 0.14 | 4.19% |
| POS1109 | 3.202 | 0.389 | 12.10% | POS1109 | 1.817 | 0.663 | 36.47% | POS1109 | 1.817 | 0.112 | 6.15% |
| STD1262 | 2.914 | 0.224 | 7.70% | STD1262 | 1.778 | 0.57 | 32.06% | STD1262 | 1.778 | 0.106 | 5.94% |
| STD1253 | 0.518 | 0.088 | 16.90% | STD1253 | 0.286 | 0.132 | 46.02% | STD1253 | 0.286 | 0.022 | 7.72% |
| STD1259 | 0.836 | 0.093 | 11.20% | STD1259 | 0.466 | 0.198 | 42.54% | STD1259 | 0.466 | 0.042 | 8.92% |
| sSyph-02 | 4.39 | 0.473 | 10.80% | sSyph-02 | 2.493 | 0.856 | 34.33% | sSyph-02 | 2.493 | 0.151 | 6.05% |
| sSyph-06 | 3.464 | 0.441 | 12.70% | sSyph-06 | 2.014 | 0.753 | 37.39% | sSyph-06 | 2.014 | 0.086 | 4.27% |
| sSyph-10 | 4.734 | 0.586 | 12.40% | sSyph-10 | 2.629 | 0.987 | 37.53% | sSyph-10 | 2.629 | 0.115 | 4.389% |
| sSyph-16 | 3.678 | 0.502 | 13.60% | sSyph-16 | 2.398 | 0.862 | 35.93% | sSyph-16 | 2.398 | 0.39 | 16.27% |
| sSyph-21 | 2.822 | 0.321 | 11.40% | sSyph-21 | 1.813 | 0.65 | 35.85% | sSyph-21 | 1.813 | 0.33 | 18.22% |

Due to the fact that the negative samples yielded small index values and that in general small values are associated with a large variation, it was reasoned that the CV % for the negative samples remained acceptable for the Syphilis assay. As for the positive samples, after an equivocal zone of index 0.47-0.80 was introduced to this assay, no positive samples tested negative or vice versa, even with a 45% of deviation. Therefore, it was concluded that the Inter-day CV % of the positive samples was also acceptable for the Syphilis assay.

Results and discussion for Adjustment of the DBS Cutoff and Accuracy. A total of 892 patient-collected DBS samples were tested; 135 of these were positive, or equivocal, according to the final reference results and 757 were negative. The package insert for the Syphilis EIA determined the result by comparing the sample Index value (Sample OD/Average OD of the low filter controls) to two constant cutoff values, the upper-cutoff and the lower-cutoff. For serum/plasma samples the upper-cutoff was set to 1.1 and lower-cutoff was set to 0.9.
If the Index upper cutoff (1.1), the test result is considered as positive for Syphilis.
If the Index lower cutoff (0.9), the test result is considered as negative for Syphilis.
If lower-cutoff (0.9)<the Index <upper-cutoff (1.1), the test result is equivocal.

The intent was to maintain the overall strategy for defining positive, equivocal, and negative samples; therefore, various combinations of upper-cutoffs and lower-cutoffs were applied to achieve the best sensitivity and specificity for the Syphilis assay. From the stability test, it was determined that the DBS samples are stable for 8 weeks at room temperature; therefore, cutoffs were calculated separately for samples tested within 8 weeks vs. all samples. Additionally, the cross-reactivity of HIV+ samples run on the Syphilis EIA was noted to be a significant factor (refer to 4.4.7); therefore, HIV− samples were also separated from the total samples. Overall, there were four distinct scenarios in which the sensitivity and specificity of potential cutoffs were calculated: 1) all samples tested (Table 4-4, n=892); 2) all samples tested within 8 weeks of generation (Table 4-5, n=449); 3) all HIV− samples tested (Table 4-7, n=469); and 4) HIV− samples tested within 8 weeks of generation (Table 4-8, n=144). For each scenario, the sensitivity and specificity for the DBS samples was calculated based on the application of various upper-cutoff and lower-cutoff values.

TABLE 4-4
The statistical measures for various upper-cutoffs and lower-
cutoffs for all samples tested on the Syphilis EIA (n = 892).

| Lower | Upperscutoff | | | | | | | | | | | Performance |
|---|---|---|---|---|---|---|---|---|---|---|---|---|
| Cutoff | 0.50 | 0.55 | 0.60 | 0.65 | 0.70 | 0.75 | 0.80 | 0.85 | 0.90 | 0.95 | 1.00 | Characteristics |
| 0.5 | 91.85 | 91.60 | 91.47 | 91.41 | 91.06 | 90.76 | 90.68 | 90.43 | 90.35 | 90.09 | 91.85 | Sensitivity % |
| | 84.15 | 86.67 | 88.72 | 91.13 | 92.45 | 93.95 | 95.65 | 96.22 | 96.52 | 97.25 | 84.15 | Specificity % |
| | 0.00 | 2.91 | 5.04 | 7.29 | 8.97 | 10.65 | 12.11 | 12.89 | 13.23 | 14.13 | 0.00 | % Equivocal |
| 0.49 | 91.85 | 91.60 | 91.47 | 91.41 | 91.06 | 90.76 | 90.68 | 90.43 | 90.35 | 90.09 | 91.85 | Sensitivity % |
| | 84.08 | 86.61 | 88.67 | 91.09 | 92.42 | 93.93 | 95.63 | 96.21 | 96.50 | 97.24 | 84.08 | Specificity % |
| | 0.34 | 3.25 | 5.38 | 7.62 | 9.30 | 10.99 | 12.44 | 13.23 | 13.57 | 14.46 | 0.34 | % Equivocal |
| 0.48 | 93.23 | 93.02 | 92.91 | 92.86 | 92.56 | 92.31 | 92.24 | 92.04 | 91.96 | 91.74 | 93.23 | Sensitivity % |
| | 83.96 | 86.50 | 88.58 | 91.01 | 92.35 | 93.87 | 95.59 | 96.17 | 96.47 | 97.21 | 83.96 | Specificity % |
| | 1.23 | 4.15 | 6.28 | 8.52 | 10.20 | 11.88 | 13.34 | 14.13 | 14.46 | 15.36 | 1.23 | % Equivocal |
| 0.47 | 93.94 | 93.75 | 93.65 | 93.60 | 93.33 | 93.10 | 93.04 | 92.86 | 92.79 | 92.59 | 93.94 | Sensitivity % |
| | 83.81 | 86.37 | 88.46 | 90.92 | 92.27 | 93.81 | 95.54 | 96.13 | 96.43 | 97.18 | 83.81 | Specificity % |
| | 2.13 | 5.04 | 7.17 | 9.42 | 11.10 | 12.78 | 14.24 | 15.02 | 15.36 | 16.26 | 2.13 | % Equivocal |
| 0.46 | 93.94 | 93.75 | 93.65 | 93.60 | 93.33 | 93.10 | 93.04 | 92.86 | 92.79 | 92.59 | 93.94 | Sensitivity % |
| | 83.65 | 86.24 | 88.35 | 90.83 | 92.19 | 93.74 | 95.49 | 96.09 | 96.39 | 97.15 | 83.65 | Specificity % |
| | 2.91 | 5.83 | 7.96 | 10.20 | 11.88 | 13.57 | 15.02 | 15.81 | 16.14 | 17.04 | 2.91 | % Equivocal |
| 0.45 | 93.94 | 93.75 | 93.65 | 93.60 | 93.33 | 93.10 | 93.04 | 92.86 | 92.79 | 92.59 | 93.94 | Sensitivity % |
| | 83.54 | 86.14 | 88.26 | 90.76 | 92.13 | 93.69 | 95.45 | 96.06 | 96.36 | 97.13 | 83.54 | Specificity % |
| | 3.48 | 6.39 | 8.52 | 10.76 | 12.44 | 14.13 | 15.58 | 16.37 | 16.70 | 17.60 | 3.48 | % Equivocal |

TABLE 4-5
The statistical measures for various upper-cutoffs and lower-cutoffs for all
samples tested on the Syphilis EIA within 8 weeks of generation (n = 449).

| Lower | Upper-cutoff | | | | | | | | | | | Performance |
|---|---|---|---|---|---|---|---|---|---|---|---|---|
| Cutoff | 0.50 | 0.55 | 0.60 | 0.65 | 0.70 | 0.75 | 0.80 | 0.85 | 0.90 | 0.95 | 1.00 | Characteristics |
| 0.50 | 92.45 | 92.16 | 92.00 | 91.92 | 91.49 | 91.30 | 91.21 | 90.91 | 90.80 | 90.59 | 90.24 | Sensitivity % |
| | 83.97 | 86.49 | 88.07 | 90.57 | 91.72 | 93.51 | 94.74 | 95.05 | 95.05 | 96.32 | 96.97 | Specificity % |
| | 0.00 | 3.12 | 4.90 | 7.13 | 9.13 | 10.91 | 12.03 | 12.92 | 13.14 | 14.48 | 15.59 | % Equivocal |
| 0.49 | 92.45 | 92.16 | 92.00 | 91.92 | 91.49 | 91.30 | 91.21 | 90.91 | 90.80 | 90.59 | 90.24 | Sensitivity % |
| | 83.92 | 86.45 | 88.04 | 90.54 | 91.69 | 93.49 | 94.72 | 95.03 | 95.03 | 96.31 | 96.96 | Specificity % |
| | 0.22 | 3.34 | 5.12 | 7.35 | 9.35 | 11.14 | 12.25 | 13.14 | 13.36 | 14.70 | 15.81 | % Equivocal |
| 0.48 | 94.23 | 94.00 | 93.88 | 93.81 | 93.48 | 93.33 | 93.26 | 93.02 | 92.94 | 92.77 | 92.50 | Sensitivity % |
| | 83.82 | 86.36 | 87.96 | 90.48 | 91.64 | 93.44 | 94.68 | 95.00 | 95.00 | 96.28 | 96.94 | Specificity % |
| | 1.11 | 4.23 | 6.01 | 8.24 | 10.24 | 12.03 | 13.14 | 14.03 | 14.25 | 15.59 | 16.70 | % Equivocal |
| 0.47 | 95.15 | 94.95 | 94.85 | 94.79 | 94.51 | 94.38 | 94.32 | 94.12 | 94.05 | 93.90 | 93.67 | Sensitivity % |
| | 83.73 | 86.28 | 87.89 | 90.42 | 91.59 | 93.40 | 94.65 | 94.97 | 94.97 | 96.26 | 96.92 | Specificity % |
| | 1.78 | 4.90 | 6.68 | 8.91 | 10.91 | 12.69 | 13.81 | 14.70 | 14.92 | 16.26 | 17.37 | % Equivocal |
| 0.46 | 95.15 | 94.95 | 94.85 | 94.79 | 94.51 | 94.38 | 94.32 | 94.12 | 94.05 | 93.90 | 93.67 | Sensitivity % |
| | 83.58 | 86.15 | 87.77 | 90.32 | 91.50 | 93.33 | 94.59 | 94.92 | 94.92 | 96.22 | 96.89 | Specificity % |
| | 2.45 | 5.57 | 7.35 | 9.58 | 11.58 | 13.36 | 14.48 | 15.37 | 15.59 | 16.93 | 18.04 | % Equivocal |
| 0.45 | 95.15 | 94.95 | 94.85 | 94.79 | 94.51 | 94.38 | 94.32 | 94.12 | 94.05 | 93.90 | 93.67 | Sensitivity % |
| | 83.58 | 86.15 | 87.77 | 90.32 | 91.50 | 93.33 | 94.59 | 94.92 | 94.92 | 96.22 | 96.89 | Specificity % |
| | 2.45 | 5.57 | 7.35 | 9.58 | 11.58 | 13.36 | 14.48 | 15.37 | 15.59 | 16.93 | 18.04 | % Equivocal |

TABLE 4-6
Comparison of in-house and reference lab Syphilis test results
for all DBS samples tested within 8 weeks of generation.

MTL

| Reference | Positive | Negative | Equivocal | Total |
|---|---|---|---|---|
| Positive | 83 | 5 | 18 | 106 |
| Negative | 16 | 283 | 44 | 343 |
| Total | 99 | 288 | 62 | 449 |

TABLE 4-7
The statistical measures for various upper-cutoffs and lower-cutoffs for all HIV-samples tested on the Syphilis EIA (n = 469).

| Lower | Upper-cutoff | | | | | | | | | | | Performance |
|---|---|---|---|---|---|---|---|---|---|---|---|---|
| Cutoff | 0.50 | 0.55 | 0.60 | 0.65 | 0.70 | 0.75 | 0.80 | 0.85 | 0.90 | 0.95 | 1.00 | Characteristics |
| 0.50 | 100.00 | 100.00 | 100.00 | 100.00 | 100.00 | 100.00 | 100.00 | 100.00 | 100.00 | 100.00 | 100.00 | Sensitivity % |
| | 88.05 | 90.87 | 92.56 | 94.76 | 95.67 | 96.37 | 97.79 | 98.27 | 98.51 | 98.76 | 99.00 | Specificity % |
| | 0.00 | 2.99 | 4.69 | 6.82 | 7.89 | 8.53 | 9.81 | 10.23 | 10.45 | 10.66 | 10.87 | % Equivocal |
| 0.49 | 100.00 | 100.00 | 100.00 | 100.00 | 100.00 | 100.00 | 100.00 | 100.00 | 100.00 | 100.00 | 100.00 | Sensitivity % |
| | 88.05 | 90.87 | 92.56 | 94.76 | 95.67 | 96.37 | 97.79 | 98.27 | 98.51 | 98.76 | 99.00 | Specificity % |
| | 0.00 | 2.99 | 4.69 | 6.82 | 7.89 | 8.53 | 9.81 | 10.23 | 10.45 | 10.66 | 10.87 | % Equivocal |
| 0.48 | 100.00 | 100.00 | 100.00 | 100.00 | 100.00 | 100.00 | 100.00 | 100.00 | 100.00 | 100.00 | 100.00 | Sensitivity % |
| | 87.95 | 90.78 | 92.49 | 94.71 | 95.63 | 96.33 | 97.77 | 98.25 | 98.50 | 98.75 | 98.99 | Specificity % |
| | 0.85 | 3.84 | 5.54 | 7.68 | 8.74 | 9.38 | 10.66 | 11.09 | 11.30 | 11.51 | 11.73 | % Equivocal |
| 0.47 | 100.00 | 100.00 | 100.00 | 100.00 | 100.00 | 100.00 | 100.00 | 100.00 | 100.00 | 100.00 | 100.00 | Sensitivity % |
| | 87.78 | 90.65 | 92.38 | 94.63 | 95.57 | 96.28 | 97.73 | 98.23 | 98.48 | 98.73 | 98.98 | Specificity % |
| | 2.13 | 5.12 | 6.82 | 8.96 | 10.02 | 10.66 | 11.94 | 12.37 | 12.58 | 12.79 | 13.01 | % Equivocal |
| 0.46 | 100.00 | 100.00 | 100.00 | 100.00 | 100.00 | 100.00 | 100.00 | 100.00 | 100.00 | 100.00 | 100.00 | Sensitivity % |
| | 87.67 | 90.57 | 92.31 | 94.58 | 95.52 | 96.24 | 97.71 | 98.21 | 98.46 | 98.71 | 98.97 | Specificity % |
| | 2.99 | 5.97 | 7.68 | 9.81 | 10.87 | 11.51 | 12.79 | 13.22 | 13.43 | 13.65 | 13.86 | % Equivocal |
| 0.45 | 100.00 | 100.00 | 100.00 | 100.00 | 100.00 | 100.00 | 100.00 | 100.00 | 100.00 | 100.00 | 100.00 | Sensitivity % |
| | 87.59 | 90.50 | 92.25 | 94.54 | 95.49 | 96.21 | 97.69 | 98.20 | 98.45 | 98.70 | 98.96 | Specificity % |
| | 3.62 | 6.61 | 8.32 | 10.45 | 11.51 | 12.15 | 13.43 | 13.86 | 14.07 | 14.29 | 14.50 | % Equivocal |

TABLE 4-8
The statistical measures for various upper-cutoffs and lower-cutoffs for all
HIV-samples tested on the Syphilis EIA within 8 weeks of generation (n = 144).

| Lower | Upper-cutoff | | | | | | | | | | | Performance |
|---|---|---|---|---|---|---|---|---|---|---|---|---|
| Cutoff | 0.50 | 0.55 | 0.60 | 0.65 | 0.70 | 0.75 | 0.80 | 0.85 | 0.90 | 0.95 | 1.00 | Characteristics |
| 0.50 | 100.00 | 100.00 | 100.00 | 100.00 | 100.00 | 100.00 | 100.00 | 100.00 | 100.00 | 100.00 | 100.00 | Sensitivity % |
| | 93.28 | 95.42 | 95.42 | 96.90 | 96.90 | 97.66 | 97.66 | 97.66 | 97.66 | 98.43 | 98.43 | Specificity % |
| | 0.00 | 2.08 | 2.08 | 3.47 | 4.17 | 4.86 | 4.86 | 4.86 | 4.86 | 5.56 | 5.56 | % Equivocal |
| 0.49 | 100.00 | 100.00 | 100.00 | 100.00 | 100.00 | 100.00 | 100.00 | 100.00 | 100.00 | 100.00 | 100.00 | Sensitivity 9% |
| | 93.28 | 95.42 | 95.42 | 96.90 | 96.90 | 97.66 | 97.65 | 97.66 | 97.66 | 98.43 | 98.43 | Specificity % |
| | 0.00 | 2.08 | 2.08 | 3.47 | 4.17 | 4.86 | 4.86 | 4.86 | 4.86 | 5.56 | 5.56 | % Equivocal |
| 0.48 | 100.00 | 100.00 | 100.00 | 100.00 | 100.00 | 100.00 | 100.00 | 100.00 | 100.00 | 100.00 | 100.00 | Sensitivity % |
| | 93.28 | 95.42 | 95.42 | 96.90 | 96.90 | 97.66 | 97.66 | 97.66 | 97.66 | 98.43 | 98.43 | Specificity % |
| | 0.00 | 2.08 | 2.08 | 3.47 | 4.17 | 4.86 | 4.86 | 4.86 | 4.86 | 5.56 | 5.56 | % Equivocal |
| 0.47 | 100.00 | 100.00 | 100.00 | 100.00 | 100.00 | 100.00 | 100.00 | 100.00 | 100.00 | 100.00 | 100.00 | Sensitivity % |
| | 93.23 | 95.38 | 95.38 | 96.88 | 96.88 | 97.64 | 97.64 | 97.64 | 97.64 | 98.41 | 98.41 | Specificity % |
| | 0.69 | 2.78 | 2.78 | 4.17 | 4.86 | 5.56 | 5.56 | 5.56 | 5.56 | 6.25 | 6.25 | % Equivocal |
| 0.46 | 100.00 | 100.00 | 100.00 | 100.00 | 100.00 | 100.00 | 100.00 | 100.00 | 100.00 | 100.00 | 100.00 | Sensitivity % |
| | 93.18 | 95.35 | 95.35 | 96.85 | 96.85 | 97.62 | 97.62 | 97.62 | 97.62 | 98.40 | 98.40 | Specificity % |
| | 1.39 | 3.47 | 3.47 | 4.86 | 5.56 | 6.25 | 6.25 | 6.25 | 6.25 | 6.94 | 6.94 | % Equivocal |
| 0.45 | 100.00 | 100.00 | 100.00 | 100.00 | 100.00 | 100.00 | 100.00 | 100.00 | 100.00 | 100.00 | 100.00 | Sensitivity % |
| | 93.18 | 95.35 | 95.35 | 96.85 | 96.85 | 97.62 | 97.62 | 97.62 | 97.62 | 98.40 | 98.40 | Specificity % |
| | 1.39 | 3.47 | 3.47 | 4.86 | 5.56 | 6.25 | 6.25 | 6.25 | 6.25 | 6.94 | 6.94 | % Equivocal |

Because the HIV+ rate is very low in the general population, it is better to use the data from all HIV− samples tested within 8 weeks after generation to calculate the new cutoffs. Indeed, these samples achieved excellent sensitivity and specificity (Table 4-8). However, since the total number of samples (n=144) and the number of Syphilis positive samples in this group are small (n=10), the upper-cutoff and lower-cutoff can be set to a wide range of values. To narrow down the selection range for the cutoff the second to best data set, containing all samples tested within 8 weeks (refer to Table 4-5), was used.

When the upper-cutoff was set to 0.80 and the lower-cutoff was set to 0.47, a sensitivity of 94.44% and a specificity of 94.97% was achieved. With these cutoffs, about 13.78% of the samples fall into the equivocal zone (Table 4-5 and Table 4-6). Additionally, these cutoffs also achieved the best sensitivity/specificity combination in other data sets.

Results and discussion for Linearity. The Syphilis assay revealed linear signals in the range from 0.05× to 1× concentration of the low titer control, resulting in an R-squared value of 0.9747 for the linear regression. See Table 4-9 and graph below. Because the assay still provides linear signals across the equivocal range for DBS samples, it was concluded that using lower Index values as cutoffs is a valid means of defining positive, negative, and equivocal samples.

TABLE 4-9
OD results of serial dilutions of Syphilis Low Titer Control.

| Dilution Ratio | 0.05 | 0.2 | 0.4 | 0.6 | 0.8 | 1 |
|---|---|---|---|---|---|---|
| Experiment 1 | 0.023 | 0.122 | 0.185 | 0.273 | 0.388 | 0.404 |
| Experiment 2 | 0.015 | 0.101 | 0.186 | 0.299 | 0.34 | 0.375 |
| Experiment 3 | 0.018 | 0.115 | 0.162 | 0.272 | 0.366 | 0.386 |
| OD Average | 0.019 | 0.113 | 0.184 | 0.281 | 0.365 | 0.388 |
| ODSD | 0.004 | 0.011 | 0.002 | 0.015 | 0.024 | 0.015 |

Results and Discussion for Stability. Nine DBS samples were tested over a period of 8 weeks. Regression analysis showed that three out of the 12 samples exhibited significantly decreased test signals within 8 weeks. See Table 4-10 and 4-11. The decrease in signal after eight weeks was roughly 30% of the original signals. While the majority of the DBS samples are stable for at least 8-week post-generation, it was concluded that samples should be tested as soon as possible for Syphilis assay. Caution should also be taken when interpreting test results coming from 8+ week old samples.

TABLE 4-10
Index values for patient DBS samples tested on the Syphilis assay for stability purposes.

| | Samples | | | | | | |
|---|---|---|---|---|---|---|---|
| Week | PSHS1052 | POS1052 | POS1143 | POS1177 | POS1179 | POS1181 | POS1182 |
| 0 | 3.300 | 3.221 | 2.080 | 2.360 | 1.540 | 2.000 | 1.130 |
| 1 | 3.948 | 3.506 | 1.570 | 2.340 | 1.290 | 0.860 | 1.820 |
| 2 | 3.492 | 3.388 | 1.260 | 1.721 | 1.259 | 1.466 | 0.893 |
| 4 | 2.819 | 2.652 | 1.110 | 2.625 | 1.436 | 2.120 | 1.151 |
| 6 | 2.717 | 2.457 | 1.571 | 2.709 | 1.444 | 2.062 | 1.212 |
| 8 | 2.763 | 2.659 | 1.825 | 2.327 | 1.554 | 2.008 | 1.036 |
| p-value | 0.061 | 0.040 | 0.891 | 0.497 | 0.394 | 0.302 | 0.536 |
| % Decrease | — | 28.1% | — | — | — | — | — |

TABLE 4-11
Index values for synthetic DBS samples tested
on the Syphilis assay for stability purposes.

| | Samples | | | | |
|---|---|---|---|---|---|
| Week | sSyph-02 | sSyph-06 | sSyph-10 | sSyph-16 | sSyph-21 |
| 0 | 2.918 | 2.318 | 3.055 | 2.367 | 2.010 |
| 1 | 2.953 | 2.528 | 3.032 | 2.287 | 1.670 |
| 2 | 2.878 | 2.401 | 2.745 | 2.288 | 1.702 |
| 5 | 2.670 | 2.270 | 3.105 | 2.560 | 1.829 |
| 6 | 2.326 | 1.624 | 2.430 | 1.898 | 1.373 |
| 8 | 2.342 | 1.758 | 2.309 | 2.003 | 1.679 |
| p-value | 0.005 | 0.040 | 0.086 | 0.267 | 0.280 |
| % Decrease | 22.9% | 31.2% | — | — | — |

Results and discussion for Carryover. To determine the possibility of using one puncher to punch multiple separate sample cards, the influence (carryover) of high-positive patient samples to subsequently punched negative patient samples was tested. This experiment revealed that after generating 20 high-positive punches, i.e., punching five unique patient samples four times each, no carryover was identified. The relative signal changes of the test punches compared to the negative cards varied from sample to sample (Table 4-13). None of the negative samples punched after the high positives resulted in a significant increase in comparison to the original negative sample signal, refer to Table 4-12.

TABLE 4-12
Average Index values for all DBS samples tested in the carryover experiment.

| Positive | Positive | Negative | Negative | Test | Test | Test | Test | Test |
|---|---|---|---|---|---|---|---|---|
| Sample ID | Card OD | Sample ID | Card OD | Punch-1 | Punch-2 | Punch-3 | Punch-4 | Punch-5 |
| POS1012 | 0.818 | STD1120 | 0.064 | 0.090 | 0.115 | 0.082 | 0.074 | 0.055 |
| POS1052 | 1.026 | STD1159 | 0.097 | 0.094 | 0.096 | 0.108 | 0.105 | 0.095 |
| STD1121 | 0.925 | STD1207 | 0.111 | 0.111 | 0.113 | 0.094 | 0.101 | 0.114 |
| STD1206 | 1.072 | STD1209 | 0.069 | 0.074 | 0.078 | 0.076 | 0.060 | 0.061 |
| STD1370 | 1.010 | STD1214 | 0.096 | 0.085 | 0.066 | 0.056 | 0.073 | 0.080 |
| | | | t-test | 0.307 | 0.332 | 0.350 | 0.238 | 0.062 |

TABLE 4-13
Normalized signals for DBS samples tested in the carryover experiment.
Normalized signals were calculated as Test Punch OD/Negative Card OD

| Positive | Positive | Negative | Negative | Test | Test | Test | Test | Test |
|---|---|---|---|---|---|---|---|---|
| Sample ID | Card OD | Sample ID | Card | Punch-1 | Punch-2 | Punch-3 | Punch-4 | Punch-5 |
| POS1012 | 0.8175 | STD1120 | 1 | 1.406 | 1.797 | 1.273 | 1.156 | 0.859 |
| POS1052 | 1.0255 | STD1159 | 1 | 0.974 | 0.990 | 1.114 | 1.088 | 0.984 |
| STD1121 | 0.925 | STD1207 | 1 | 0.995 | 1.014 | 0.847 | 0.905 | 1.027 |
| STD1206 | 1.072 | STD1209 | 1 | 1.072 | 1.130 | 1.094 | 0.862 | 0.884 |
| STD1370 | 1.01 | STD1214 | 1 | 0.885 | 0.686 | 0.581 | 0.759 | 0.838 |
| | | | Average | 1.067 | 1.123 | 0.982 | 0.964 | 0.919 |

Results and discussion for Interferences. The sensitivity of the CAPTIA™ Syphilis (T. pallidum)-G EIA to interferences was tested. The influence of seven substances that are commonly encountered during DBS sample collection were focused on. Based on the established CV % from the precision study, an SD value was calculated for each sample at its signal level. See Table 4-14. If the differences between samples containing versus those without potential interfering substances were larger than ±3 SD range, they were assigned a significance score “1”, otherwise samples were assigned an “0”. As shown in Table 4-15, 1% of soap can cause significant false positive signals. Besides the solution of 1% soap, the Syphilis EIA test was not significantly affected by other substances tested, such as solutions containing 1% of ethanol, soil, hair gel, hand sanitizer, lotion, and saliva.

TABLE 4-14
Index values for Syphilis positive and negative samples tested utilizing
elution buffers with various potential interfering substances.

| Potential | | Index Value | | | | | |
|---|---|---|---|---|---|---|---|
| Interference | | Syph P1 | Syph P2 | Syph P3 | N1 | N2 | N3 |
| A | Ethanol | 2.98 | 1.810 | 0.540 | 0.160 | 0.160 | 0.360 |
| B | Soil | 2.82 | 1.880 | 0.520 | 0.170 | 0.130 | 0.310 |
| C | Hair gel | 2.75 | 2.010 | 0.450 | 0.150 | 0.170 | 0.270 |
| D | Hand sanitizer | 2.89 | 1.970 | 0.570 | 0.170 | 0.120 | 0.260 |
| E | Lotion | 2.71 | 1.850 | 0.560 | 0.240 | 0.190 | 0.350 |
| F | Saliva | 2.88 | 2.040 | 0.640 | 0.230 | 0.180 | 0.340 |
| G | Soap | OVER | 5.090 | 5.010 | 4.750 | 4.770 | OVER |
| H | N/A (Control) | 2.86 | 1.960 | 0.650 | 0.200 | 0.180 | 0.360 |

| | | | | | | |
|---|---|---|---|---|---|---|
| Precision CV %ₐ | 15.90 | 17.80 | 27.30 | 26.20 | 26.20 | 22.40 |
| Calculated SD_{b} | 0.45 | 0.35 | 0.18 | 0.05 | 0.05 | 0.08 |

ₐCV % established from Precision tests.
_{b}SD = CV %^{a}Control/100

TABLE 4-15
Significance of potential interfering substances on the Syphilis test.

| Pote ntial | | Significance | | | | | |
|---|---|---|---|---|---|---|---|
| Interference | | Syph P1 | Syph P2 | Syph P3 | N1 | N2 | N3 |
| A | Ethanbl | 0 | 0 | 0 | 0 | 0 | 0 |
| B | Soil | 0 | 0 | 0 | 0 | 0 | 0 |
| C | 8airgel | 0 | 0 | 0 | 0 | 0 | 0 |
| D | Hand sahitizer | 0 | 0 | 0 | 0 | 0 | 0 |
| E | Lotion | 0 | 0 | 0 | 0 | 0 | 0 |
| F | Saliva | 0 | 0 | 0 | 0 | 0 | 0 |
| G | Soap | 1 | 1 | 1 | 1 | 1 | 1 |
| H | N/A(Cohtrol) | 0 | 0 | 0 | 0 | 0 | 0 |

Note:
“O” indicates differences between test samples and controls were less than ±3SD, i.e. not significant. “1” indicates difference were larger than ±3SD, i.e. significant.

Results and discussion for Cross-reactivity of Syphilis assay to HIV, HCV and HSV-2. In order to determine if the Syphilis assay is cross-reactive to the other three disease types, such as HCV, HIV and HSV-2, Syphilis results were correlated to the results for the same sample on the other assays. A total of 267 samples, with reference results for all four assays, was used in this analysis.

Case 1) Syphilis' cross-reactivity to HIV signals. Among all the HSV-2− and HCV− samples, there were 185 HIV−/Syph− samples, 54 HIV+/Syph− samples, 5 HIV−/Syph+ samples, and 6 HIV+/Syph+ samples. Analysis bins were set up according to the Syphilis Index for a total range from 0.05 to 2.0. Bins for the 0.05 to 1.55 index range were separated by increments of 0.05. The remaining samples separated into a 1.55-2.0 range bin and a bin for samples with an index>2.0. The frequency of samples in each index bin were plotted against the index value. Sample distribution was comparable between HIV+/Syph− and HIV−/Syph− groups. This indicates that the Syphilis assay does not crosstalk to HIV antibody/antigens. However, HIV+/Syph+ samples generally had lower Syphilis Index than HIV−/Syph+ samples. This could be biased due to the small sample size. Another possibility is that HIV+ patients have compromised immune system, therefore generating less amount of Syphilis antibody. See figure below.

Case 2) Syphilis cross-reactivity to HCV signals. Among all the HIV− and HSV-2− samples, there were 185 HCV−/Syph− samples, 55 HCV+/Syph− samples, 6 HCV−/Syph+ samples, and 0 HCV+/Syph+ samples. Therefore, it was only possible to determine if HCV positive signals affect Syphilis background or not. Analysis bins were set up according to the Syphilis Index for a total range of 0.05 to 1.55. Each bin represents a 0.05 index increment difference. The frequency of samples in each index bin were plotted against Index values. Sample distribution was comparable between HCV+/Syph− and HCV−/Syph− groups. Therefore, HCV positive signals do not affect Syphilis background. See figure below.

Case 3) Syphilis's cross-reactivity to HSV-2 signals. Among all the HIV− and HCV− samples, there were 185 HSV-2−/Syph− samples, 72 HSV-2+/Syph− samples, 5 HSV-2−/Syph+ samples, and 5 HSV-2+/Syph+ samples. Analysis bins were set up according to the Syphilis Index for a total range of 0.05 to 2.0. Bins for the 0.05 to 1.55 index range were separated by increments of 0.05 with the remaining samples separated into a 1.55-2 range bin and a bin for samples with an index>2. The frequency of samples in each index bin were plotted against their Index value. sample distributions were comparable between HSV-2+/Syph− and HSV-2−/Syph− samples. This indicates that the Syphilis assay does not crosstalk to HSV-2 antibody/antigens. For Syphilis positive samples, even though 2 of the HSV-2+/Syph+ samples had very low Syphilis Index, the sample size was too small to make any sound conclusions. See figure below.

Results and discussion for Comparison between DSX and Semi-automatic method. Eleven positive samples (three HIV+, three HSV-2+, three HCV+, and two Syphilis+ samples) and six negative samples were used in this study. Elution of each sample was divided into two tubes: one for the DSX and the other one for the semi-automatic method. Paired student t-test was used to analyze the differences of Syphilis negative samples between the two methods. As shown in Table 4-16, all the Syphilis negative samples tested had lower signals from the semi-automatic method in comparison to the DSX method. The difference was highly significant with a p-value of 6.56\hat{ }10⁻⁷. However, the Index values of Syphilis positive samples were comparable or even higher when the semi-automatic method was used. Based on this result, we conclude that it is necessary to repeat all the positive and equivocal results generated initially by DSX with semi-automatic methods, because the latter gives much lower background.

TABLE 4-16
Comparison between DSX and Semi-automatic method.

| Reference | | Semi-Automatic (Index) | | | DSX (Index) | | |
|---|---|---|---|---|---|---|---|
| Result | Samples | Repeat 1 | Repeat2 | Average | Repeat 1 | Repeat2 | Average |
| Neg | Air Blank | 0 | | N/A | 0 | | N/A |
| Syp+ | High-Titre Control | 3.991 | | N/A | 3.600 | | N/A |
| Syp+ | Low-Titre Control | 1.007 | 0.993 | 1.000 | 1.090 | 0.910 | 1.000 |
| Neg | Non-React. Control | 0.023 | | N/A | 0.010 | | N/A |
| Syp+ | STD1263-A | 2.465 | 2.676 | 2.571 | 2.310 | 1.740 | 2.025 |
| Syp+ | POS1011-A | 1.834 | 1.636 | 1.835 | 1.330 | 1.490 | 1.410 |
| NEG | STD1471-A | 0.225 | 0.204 | 0.215 | 0.350 | 0.310 | 0.330 |
| NEG | STD1473-A | 0.191 | 0.153 | 0.172 | 0.210 | 0.210 | 0.210 |
| NEG | STD1474-A | 0.176 | 0.159 | 0.168 | 0.270 | 0.250 | 0.260 |
| NEG | STD1476-A | 0.236 | 0.191 | 0.214 | 0.380 | 0.300 | 0.340 |
| NEG | STD1479-A | 0.208 | 0.189 | 0.199 | 0.390 | 0.350 | 0.370 |
| NEG | STD1484-A | 0.132 | 0.134 | 0.133 | 0.220 | 0.240 | 0.230 |
| HIV+, SYP− | POS1249-A | 0.200 | 0.170 | 0.185 | 0.360 | 0.360 | 0.360 |
| HIV+, SYP− | POS1104-A | 0.261 | 0.242 | 0.252 | 0.590 | 0.560 | 0.575 |
| HIV+, SYP− | POS1112-A | 0.219 | 0.208 | 0.214 | 0.340 | 0.330 | 0.335 |
| HCV+, SYP− | POS1166-A | 0.283 | 0.227 | 0.255 | 0.540 | 0.440 | 0.490 |
| HCV+, SYP− | POS1155-A | 0.249 | 0.227 | 0.238 | 0.540 | 0.460 | 0.500 |
| HCV+, SYP− | STD1422-A | 0.302 | 0.302 | 0.302 | 0.510 | 0.510 | 0.510 |
| HSV2+, SYP− | STD1477-A | 0.499 | 0.476 | 0.488 | 0.650 | 0.560 | 0.605 |
| HSV2+, SYP− | POS1160-A | 0.200 | 0.196 | 0.198 | 0.440 | 0.400 | 0.420 |
| H5V2+, SYP− | STD1381-A | 0.202 | 0.196 | 0.199 | 0.370 | 0.370 | 0.370 |

| | |
|---|---|
| Student-test: | 6.560E−07 |

Note:
A cell highlighted in red indicates a “Positive” Syphilis result; a yellow cell indicates an “Equivocal” Syphilis result; and a green cell indicates a “Negative” Syphilis result.

In conclusion, the inventors have successfully validated the CAPTIA™ Syphilis (T. pallidum)-G EIA on the DSX with patient self-collected Dried Blood Spot cards and is ready for clinical implementation. When the upper-cutoff was set at 0.8 and the lower-cutoff was set at 0.47, the sensitivity and specificity of this assay were 94.32% and 94.65%, based on 450 patient samples collected on DBS cards (GE Whatman™ 903 protein saver cards). When measured by Index value, the inter-day precision exhibited a CV around 30%-40% and the intra-day precision exhibited a CV less than 20%. Most DBS samples have been shown to be stable for 8 weeks post-generation, when stored at room temperature, however 25% ( 3/12) of samples showed roughly a 30% decrease of signals after 8 weeks post-generation. Therefore, samples should be run as soon as possible. High concentration of soap can cause false positive results on the Syphilis assay, but no interference was observed from other tested substances such as 1% ethanol, soil samples, hand sanitizer, lotion, and saliva. Cross-reactive tests revealed HIV positive samples tend to decrease the signal of Syphilis positive samples, while HCV and HSV-2 positive samples do not affect background signal on the Syphilis EIA. These factors need to be taken into consideration when interpreting dual positive samples. It was also determined that the semi-automatic method exhibited a lower background signal than the DSX method. It is recommended that positive or equivocal samples be re-tested on the semi-automatic method prior to releasing any final calls.

The use of the terms “a” and “an” and “the” and similar referents in the specification and in the following claims are to be construed to cover both the singular and the plural, unless otherwise indicated herein or clearly contradicted by context. The terms “having,” “including,” “containing” and similar referents in the specification and in the following claims are to be construed as open-ended terms (e.g., meaning “including, but not limited to,”) unless otherwise noted. Recitation of ranges of values herein are merely indented to serve as a shorthand method of referring individually to each separate value inclusively falling within the range, unless otherwise indicated herein, and each separate value is incorporated into the specification as if it were individually recited herein. All methods described herein can be performed in any suitable order unless otherwise indicated herein or clearly contradicted by context. The use of any and all examples, or exemplary language (e.g., “such as”) provided herein, is intended merely to better illuminate embodiments and does not pose a limitation to the scope of the disclosure unless otherwise claimed. No language in the specification should be construed as indicating any non-claimed element as essential to each embodiment of the present disclosure.

Different arrangements of the components depicted in the drawings or described above, as well as components and steps not shown or described are possible. Similarly, some features and sub-combinations are useful and may be employed without reference to other features and sub-combinations. Embodiments have been described for illustrative and not restrictive purposes, and alternative embodiments will become apparent to readers of this patent. Accordingly, the present subject matter is not limited to the embodiments described above or depicted in the drawings, and various embodiments and modifications can be made without departing from the scope of the claims below.

That which is claimed is:

1. A method for detecting presence of sexually transmitted infection in a sample from a dried blood spot card; the method comprising:
isolating at least one sample from a dried-blood spot card having one or more dried blood spots, the isolated sample including an amount of sample material for use in an enzyme-linked immunosorbent assay system;
placing the at least one sample into an elution;
preparing the elution for an instrument for use in an enzyme-linked immunosorbent assay system;
preparing an instrument using the prepared elution;
preparing an enzyme-linked immunosorbent assay from the prepared instrument;
analyzing the enzyme-linked immunosorbent assay using the enzyme-linked immunosorbent assay system; and
determining the presence of one or more sexually transmitted infection present on the enzyme-linked immunosorbent assay.

2. The method of claim 1, wherein the preparing the elution for an instrument for use in an enzyme-linked immunosorbent assay system further comprises adding at least one reagent to the elution.

3. The method of claim 2, wherein adding at least one reagent to the elution further comprises adding BIO-RAD GS HIV Combo Ag/Ab EIA (BIO-RAD #26217).

4. The method of claim 2, wherein adding at least one reagent to the elution further comprises adding Trinity Biotech CAPTIA™ Syphilis (T. Pallidum) IgG EIA (BIO-RAD #25029).

5. The method of claim 2, wherein adding at least one reagent to the elution further comprises adding ORTHO® HCV EIA 3.0 Reagent Kit (BIO-RAD #930740).

6. The method of claim 2, wherein adding at least one reagent to the elution further comprises adding HerpeSelect™ 2 ELISA IgG (FOCUS Diagnostics #EL0920G).

7. The method of claim 2, wherein adding at least one reagent to the elution further comprises adding PBS (Phosphate Buffered Saline) 1×+0.05% Tween-20 (Growcells.com #MRGF-6275).

8. The method of claim 2, wherein adding at least one reagent to the elution further comprises adding Trinity Biotech Trep-Sure™ EIA (Trinity #TS-96).

9. The method of claim 1, wherein preparing an instrument further comprises:
depositing a first amount of elution with a first conjugate into a well on the instrument;
depositing a second amount of controls, calibrator and patient sample to the well;
incubating the instrument a first time;
washing the instrument a first time;
depositing a third amount of second conjugate to the well;
incubating the instrument a second time;
washing the instrument a second time;
depositing a fourth amount of a working substrate solution to the well;
incubating the instrument a third time;
washing the instrument a third time;
reading an optical density value of the well through a filter; and
determining the presence of HIV is the optical density is above a threshold optical density.

10. The method of claim 1, wherein preparing an instrument further comprises:
depositing a first amount of elution with a first conjugate into a well on the instrument;
depositing a second amount of controls, calibrator and patient sample to the well;
incubating the instrument a first time;
washing the instrument a first time;
depositing a third amount of second conjugate to the well;
incubating the instrument a second time;
washing the instrument a second time;
depositing a fourth amount of a working substrate solution to the well;
incubating the instrument a third time;
depositing a fifth amount of stop solution to the well; and
wherein the analyzing further comprises:
reading an optical density value of the well through a filter; and
determining the presence of HCV is the optical density is above a threshold optical density.

11. The method of claim 1, wherein preparing an instrument further comprises:
depositing a first amount of elution with a first conjugate into a well on the instrument;
depositing a second amount of controls, calibrator and patient sample to the well;
incubating the instrument a first time;
washing the instrument a first time;
depositing a third amount of second conjugate to the well;
incubating the instrument a second time;
washing the instrument a second time;
depositing a fourth amount of a working substrate solution to the well;
incubating the instrument a third time;
depositing a fifth amount of stop solution to the well; and
wherein the analyzing further comprises:
reading an optical density value of the well through a filter; and
determining the presence of HCV is the optical density is above a threshold optical density.

12. The method of claim 1, wherein preparing an instrument further comprises:
depositing a first amount of controls and patient sample to the well;
incubating the instrument a first time;
washing the instrument a first time;
depositing a second amount of an enzyme conjugate to the well;
incubating the instrument a second time;
washing the instrument a second time;
depositing a third amount of a TMB substrate solution to the well;
incubating the instrument a third time;
depositing a fourth amount of stop solution to the well; and
wherein the analyzing further comprises:
reading an optical density value of the well through a filter; and
determining the presence of HSV-2 is the optical density is above a threshold optical density.

13. The method of claim 1, wherein preparing an instrument further comprises:
depositing a first amount of controls, calibrator and patient sample to the well;
incubating the instrument a first time;
washing the instrument a first time;
depositing a second amount of an enzyme conjugate to the well;
incubating the instrument a second time;
washing the instrument a second time;
depositing a third amount of a substrate solution to the well;
incubating the instrument a third time;
depositing a fourth amount of stop solution to the well; and
wherein the analyzing further comprises:
reading an optical density value of the well through a filter; and
determining the presence of syphilis is the optical density is above a threshold optical density.

14. The method of claim 1, further comprising collecting at least one dried-blood spot remotely using a dried-blood spot card.

15. The method of claim 1, wherein detecting one or more sexually transmitted infection present on the enzyme-linked immunosorbent assay further comprises detecting the presence of one or more of the group composed of: chlamydia, gonorrhea HIV, HCV, HbsAg, syphilis, and HSV-2.

16. A system for collecting samples and determining the presence of STI in blood samples, the system comprising:
a dried-blood spot collection card configured to collect blood samples from a human patient in a setting that is remote from an analysis laboratory;
a chip-punching device configured to punch chips at the analysis laboratory from a dried-blood spot collection card after blood samples have been collected remotely and received at the analysis laboratory;
an elution preparation station configured to receive the punched chips into an elution and configured to add one or more reagents to the elution in preparation for an enzyme-linked immunosorbent assay;
an instrument preparation station configured to assemble the prepared elution onto an instrument for use in an enzyme-linked immunosorbent assay; and
an enzyme-linked immunosorbent assay station configured to analyze the prepared enzyme-linked immunosorbent instrument and configured to determine the presence of one or more sexually transmitted infections on the assay.

17. The system of claim 16, wherein elution preparation station is further configured to add at least one reagent to the elution from the group composed of: BIO-RAD GS HIV Combo Ag/Ab EIA (BIO-RAD #26217); Trinity Biotech CAPTIA™ Syphilis (T. Pallidum) IgG EIA (BIO-RAD #25029); ORTHO® HCV EIA 3.0 Reagent Kit (BIO-RAD #930740); HerpeSelect™ 2 ELISA IgG (FOCUS Diagnostics #EL0920G), Trinity Biotech Trep-Sure™ EIA (Trinity #TS-96), and PBS (Phosphate Buffered Saline) 1×+0.05% Tween-20 (Growcells.com #MRGF-6275).

18. The system of claim 16, wherein elution preparation station is further configured to rock or incubate the elution for a period of time.

19. The system of claim 16, wherein the enzyme-linked immunosorbent assay station is configured to detect the presence of one or more of the group composed of: chlamydia, gonorrhea HIV, HCV, HbsAg, syphilis, and HSV-2.

20. An assay for determining presence of one or more sexually-transmitted infections from an elution prepared from a remotely collected dried-blood sample, the assay comprising:
a microplate;
an elution deposited onto the microplate, the elution comprising:
at least material from one dried blood sample chip from a remotely collected dried blood sample;
at least one reagent; and
at least one calibrator or conjugate having at least one quality control parameter for analyzing presence of a sexually transmitted infection detectable from the dried blood spot.

21. The assay of claim 20; wherein the added reagent comprises a reagent from the group composed of: BIO-RAD GS HIV Combo Ag/Ab EIA (BIO-RAD #26217); Trinity Biotech CAPTIA™ Syphilis (T. Pallidum) IgG EIA (BIO-RAD #25029); ORTHO® HCV EIA 3.0 Reagent Kit (BIO-RAD #930740); HerpeSelect™ 2 ELISA IgG (FOCUS Diagnostics #EL0920G), Trinity Biotech Trep-Sure™ EIA (Trinity #TS-96), and PBS (Phosphate Buffered Saline) 1×+0.05% Tween-20 (Growcells.com #MRGF-6275).
